(19) United States
(12) Patent Application Publication
(10) Pub. No.: US 2023/0190895 A1
(43) Pub. Date: Jun. 22, 2023

(54) NEGATIVELY CHARGED PARTICLES FOR THE TREATMENT OF CYTOKINE STORM SYNDROME (CSS) AND ACUTE RESPIRATORY DISTRESS SYNDROME (ARDS)

(71) Applicant: onCOUR Pharma, Inc., Northbrook, IL (US)

(72) Inventors: John PUISIS, Northbrook, IL (US); Jim HERRMANN, Northbrook, IL (US)

(73) Assignee: onCOUR Pharma, Inc., Northbrook, IL (US)

(21) Appl. No.: 17/922,484

(22) PCT Filed: Apr. 29, 2021
(86) PCT No.: PCT/US21/29893

§ 371 Date: Oct. 31, 2022

Related U.S. Application Data
(60) Provisional application No. 63/018,210, filed on Apr. 30, 2020.
(60) Provisional application No. 63/018,214, filed on Apr. 30, 2020.
(60) Provisional application No. 63/128,386, filed on Dec. 21, 2020.

(51) Int. Cl. A61K 39/00; A61P 31/14

(52) U.S. Cl. CPC A61K 39/00; A61P 31/14; A61K 2039/5158

(57) ABSTRACT

Provided herein are compositions that comprise negatively charged particles and methods of making and using the same. Also provided are methods of reducing or treating Cytokine Storm Syndrome (CSS) or Acute Rcspiratoiy Distress Syndrome (ARDS).

### CROSS REFERENCE TO RELATED APPLICATIONS

This application claims the benefit of U.S. Provisional Application Nos. U.S.63/018,210, filed Apr. 30, 2020; U.S.63/018,214, filed Apr. 30, 2020; and U.S.63/128,386, filed Dec. 21, 2020 all of which are herein incorporated by reference in their entirety.

### BACKGROUND

Cytokine Storm Syndrome (CSS) and Acute Respiratory Distress Syndrome (ARDS) are potentially terminal clinical conditions driven by a cascade of inflammatory events leading to overwhelming systemic inflammation, multiorgan dysfunction, and even death. Despite some differences in clinical manifestations and triggering events, CSS and ARDS are unified by involvement of common inflammatory processes such as dysregulated activation, expansion, and functioning of innate immune cells (e.g monocytes, neutrophils, and macrophages), secretion of excessive cytokines, chemokines, and pro-inflammatory mediators which drive systemic inflammation leading to multi-organ dysfunction and mortality ¹⁻⁵. Moreover, there is significant overlap between these conditions as CSS can lead to ARDS, and vice versa, in many subjects.

CSS can occur due to a variety of diverse triggers such as viral infections, bacterial infections, pathogens, traumatic injuries, and immune-directed therapies (e.g CAR-Ts, antibodies, and cytokines). CSS is also associated with autoimmune and rheumatic conditions (e.g arthritis and lupus), macrophage activation syndrome (MAS), reactive hemophagocytic syndrome, and secondary hemophagocytic lymphohistiocytosis (sHLH). Monocytes, macrophages, and neutrophils are major drivers of CSS. The initial event triggering CSS results in the activation and expansion of inflammatory monocytes, macrophages and neutrophils via cytokine, chemokine, and growth-factory signaling. These cells are actively recruited to sites of ongoing inflammation where they respond to the pro-inflammatory milieu by producing pro-inflammatory cytokines and chemokines (e.g IL-6, IL-1β, IFN-γ, IP-10, TNF-α, and MCP-1, CCL-2, CXCL-1, CXCL-2, CXCL-5), oxidative species (e.g ROS), proteins (e.g c- reactive protein), proteases, and metabolites. The pro-inflammatory activities of these cells fuel an uncontrolled feedback loop which further escalates the inflammatory immune response causing prolonged and excessive systemic inflammation and life-threatening pathologies ⁵⁻⁸.

ARDS can be triggered by direct or indirect lung injury. Examples of ARDS resulting from direct lung injury include pneumonia due to bacterial, viral, fungal, or opportunistic infections, pulmonary contusions, traumatic injuries, inhalation injury from chemicals, particulates, or other irritants, aspirations of gastric contents, and near drowning. Examples of ARDS resulting from indirect lung injury include hemorrhagic shock, pancreatitis, major burn injury, drug overdose, transfusion of blood products, cardiopulmonary bypass, sepsis, and reperfusion injury. The initial insult triggering ARDS, whether due to direct or indirect lung injury, causes pulmonary damage that elicits a robust and heightened immune response. This immune response involves activation of resident immune cells (e.g., bronchoalveolar macrophages) that produce pro-inflammatory cytokines and chemokines immediately followed by the rapid influx of inflammatory monocytes and neutrophils into the lungs within 24 to 48 hours of initial injury. Once at the lungs, peripherally derived inflammatory monocytes and neutrophils respond to the local inflammatory milieu and further promote inflammation via production of pro-inflammatory cytokines (e.g., IL-6, IL-β, IFN-γ, IP-10, TNF-α, and MCP-1), chemokines (e.g., CCL-2, CXCL- 1, CXCL-2, and CXCL-5), oxidants (e.g., ROS), proteins (e.g., c-reactive protein), neutrophil extracellular traps (NETs), and proteases (e.g MMP-9) While a certain degree of inflammation is important for the resolution of lung injury, excessive and prolonged inflammation, especially in the case of ARDS, results in significant respiratory damage and is associated with life-threatening pathologies^{1,2,4}.

Current approaches for the treatment of CSS and ARDS rely on broad immune suppressants (e.g anti-IL1β, anti-IL-6, anti-TNFα, and steroids) which cause toxic side effects leading to increased risk of infections and even death. There is an urgent need for targeted therapies for resolving pathological hyperinflammation during CSS and ARDS without causing broad immune suppression.

### SUMMARY

The present disclosure relates to a method of treating cytokine storm syndrome (CSS) and/or acute respiratory distress syndrome (ARDS) in a subject, the method comprising administering to the subject negatively charged particles having a negative zeta potential, wherein the negatively charged particles are free from another therapeutic agent. In one embodiment of the method disclosed herein, the subject suffers from CSS and/or ARDS resulting from one or more conditions selected from: a viral infection, a bacterial infection, a fungal infection, an opportunistic infection, sepsis, cytokine release syndrome (CRS), severe inflammatory response syndrome (SIRS), hypercytokinemia, macrophage activation syndrome (MAS), reactive hemophagocytic syndrome, secondary hemophagocytic lymphohistiocytosis (sHLH), aspiration of gastric contents, a traumatic injury, burn injury, pancreatitis, pulmonary contusion, hemorrhagic shock, near drowning, blood transfusion, inhalation injury, or combinations thereof.

In any one of the methods disclosed herein, administering the negatively charged particles in the subject reduces one or more symptoms of CSS and/or ARDS. In embodiments, the symptom is selected from one or more of: multi-organ dysfunction, brain damage, lung damage, liver damage, kidney damage, heart damage, edema, cerebral edema, pulmonary edema, alveolar edema, respiratory distress, hypoxemia, respiratory acidosis, hypertriglyceridemia, leukopenia, cytopenia, or elevated levels of inflammatory markers. In any one of the methods disclosed herein, the present disclosure relates to improving one or more symptoms associated with CSS and/or ARDS, comprising administering the negatively charged particles as disclosed herein. In embodiments, the symptom associated with ARDS includes shortness of breath, rapid breathing (tachypnea), labored breathing, requiring mechanical ventilation, muscle fatigue, general fatigue, low blood pressure, low blood oxygen levels (hypoxemia), discoloration of the skin, discoloration of the nails, respiratory acidosis, hypercapnia, dry cough, fever, chest pain, headache, inflammation of the lungs, fluid buildup in the lungs, atelectasis, crackling or bubbling sound in the lungs, fast pulse rate, dizziness, mental confusion, edema, pulmonary edema, and/or alveolar edema. In embodiments, the symptom associated with CSS and/or ARDS includes one or more selected from: lung inflammation, atelectasis, distressed breathing, fatigue, low blood pressure, fever, headache, hypoxemia, respiratory acidosis, hypercapnia, edema, pulmonary edema, or alveolar edema. In embodiments, the inflammatory marker is IL-1β, IL-2, IL-6, IL-8, TNF-α, IFN-γ, MCP-1, c-reactive protein, or ferritin.

In any one of the methods disclosed herein, CSS and/or ARDS is the result of a viral infection. In embodiments, the viral infection is due to a DNA virus, an RNA virus, and/or a retrovirus. In embodiments, the DNA virus is a single-stranded DNA (ssDNA) virus, or a double-stranded (dsDNA) virus, and the RNA virus is a double-stranded RNA virus, a single-stranded RNA (ssRNA) (+) virus, ssRNA (−) virus, or a circular ssRNA virus. In embodiments, the virus is a respiratory virus. In embodiments, the virus is selected from the group consisting Adeno- associated virus, Aichi virus, Australian Bat Lyssavirus, BK polyomavirus, Banna virus, Barmah forest virus, Bunyavirus snowshoe hare, Cercopithecine herpes virus, Chandipura virus, Chikungunya virus, Cosavirus A, Cowpox virus, Coxsackievirus, Crimean-Congo hemorrhagic fever virus, Coronavirus, Dengue virus, Dhori virus, Dugbe virus, Duvenhage virus, Eastern equine encephalitis virus, Echovirus, Encephalomyocarditis virus, Epstein-Barr virus, European Bat Lyssavirus, GB virus C/Hepatitis G virus, Hantaan virus, Hendra virus, Hepatitis A virus, Hepatitis B virus, Hepatitis C virus, Hepatitis delta virus, Human adenovirus, Human astrovirus, Human coronavirus, Human cytomegalovirus, Human enterovirus 68,70, Human herpesvirus 1, Human herpesvirus 2, Human herpesvirus 6, Human herpesvirus 7, Human Immunodeficiency Virus (HIV), Human papillomavirus 1, Human papillomavirus 2, Human papillomavirus 16,18, Human parainfluenza virus, Human parvovirus B19, Human respiratory syncytial virus, Human rhinovirus, Human SARS coronavirus, Human spumarterovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro visu, MERS coronavirus, Measles virus, Mengo encephalomyocarditis, Merkell cell polyomavirus, Mokola virus, Molluscum contagiosum virus, Monkeypox virus, Mumps virus, Murray valley encephalitis virus, New York virus, Nipah virus, Norwalk virus, O'Nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama, Salivirus A, Sandfly fever sicilian virus, Sapporo virus, SARS coronavirus-2, Semliki forest virus, Seoul virus, Simian foamy virus, Simian virus 5, Sindbis virus, Southampton virus, St. Louis encephalitis virus, Tick-borne Powassan virus, Torque teno virus, Toscana virus, Uukuniemi virus, Varicella-zoster virus, Variola virus, Venezuelan equine encephalitis virus, WU polyomavirus, Yaba Monkey tumor virus, Yaba-like disease virus, Yellow fever virus, or Zika virus.

In any of the methods disclosed here, CSS and/or ARDS is the result of a bacterial infection. In embodiments, the bacterial infection is due to Staphylococcus, Streptococcus, Mycobacterium, Bacillus, Salmonella, Vibrio, Spirochete, Neisseria, Diplococcus, Pseudomonas, Clostridium, Treponaema, Spirillum, or combinations thereof.

In any of the methods disclosed here, CSS and/or ARDS is due to one or more immune targeted therapies. In embodiments, the immune targeted therapy an antibody, a protein therapeutic, a peptide, a cytokine, an immune signaling modulator, an mRNA, an oncolytic virus, or a cell-based therapy. In embodiments, the antibody is a monoclonal antibody, a polyclonal antibody, a bi-specific antibody, a tri-specific antibody, or a bi-specific T-cell engager (BiTE) antibody. In embodiments, the antibody targets one or more of: CD2, CD3, CD20, CD27, CD28, CD30, CD4OL, CD137, OX-40, GITR, LIGHT, DR3, SLAM, or ICOS. In embodiments, the cytokine is selected from IFN-a, IFN-y, IL-2, IL-10, IL-12, IL-15, IL-15/IL-15Rα, IL-18, IL-21, GM-CSF, or variants thereof. In embodiments, the immune signaling modulator targets one or more of: IL-1R, IL-2Rα, IL-2Rβ, IL-2Rγ, IL-3Rα, CSF2RB, IL-4R, IL-5Rα, CSF2RB, IL-6Rα, gp130, IL-7Rα, IL-9R, IL-10Rα, IL-10Rβ, IL-12Rβ1, IL-12Rβ2, IL-13Rα1, IL-13Rα2, IL-15Rα, IL-21R, IL23R, IL-27Rα, IL-31Rα, OSMR, CSF-1R, GM-CSF-R, cell-surface IL-15, IL-10Rα, IL-10Rβ, IL-20Rα, IL-20Rβ, IL-22Rα1, IL-22Rα2, IL-22Rβ, IL-28RA, TLR, JAK, BTK, TYK, SYK, MAPK, PI3K, NFKB, NFAT, STAT, or a kinase. In embodiments, the cell-based therapy comprises allogenic, autologous, or iPSC-derived cells. In embodiments, the cell-based therapy comprises one or more of: T-cells, NK-cells, red blood cells, stem cells, antigen-presenting cells, macrophages, or dendritic cells.

In any one of the methods disclosed herein, the negatively charged particle comprises one or more of: polyglycolic acid (PGA), polylactic acid (PLA), polystyrene, poly (lactic-co- glycolic acid) (PLGA), chitosan, polysaccharide, a lipid, diamond, iron, zinc, cadmium, gold, or silver. In embodiments, the negatively charged particle is poly (lactic-co-glycolic acid) (PLGA) particle. In embodiments, the PLGA particle comprises a ratio of poly lactic acid : poly glycolic acid ranging from about 90:10 to about 10:90, from about 50:50 to about 90:10, from about 50:50 to about 80:20; from about 90:10 to about 50:50, or from about 80:20 to about 50:50. In embodiments, the negatively charged particle comprises 50:50 poly lactic acid : poly glycolic acid.

In any one of the methods disclosed herein, the negatively charged particle is surface- functionalized by the addition of one or more carboxyl groups on the particle's surface.

In any one of the methods disclosed herein, the negatively charged particle has a zeta potential between about −100 mV and about −1 mV. In embodiments, the negatively charged particle has a zeta potential between about −80 mV and about −30 mV.

In any one of the methods disclosed herein, the negatively charged particle has a mean diameter in the range of about 0.1 μm to about 10 μm. In embodiments, the negatively charged particle has a mean diameter in the range of about 300 nm to about 800 nm.

In any one of the methods disclosed herein, the negatively charged particles are administered intravenously.

### BRIEF DESCRIPTION OF THE DRAWINGS

FIG. 1 shows the effect of ONP-302 on weight loss after primary LCMV infection. C57BL/6 mice were infected with 2×10⁶ plaque forming units (pfu) LCMV (clone 13) via intravenous tail vein injection. On Day 5 post-infection, mice were randomized into one of three treatment groups and administered the indicated treatments via tail vein injection. Mice were followed for weight loss. (n=5 per treatment group) (*=p<0.05; **=p<0.005; ***=p<0.0005; ****=p<0.00005).

FIG. 2A shows the effect of ONP-302 on immune cells in the spleen of mice infected with LCMV. C57BL/6 mice were infected with 2×10⁶ plaque forming units (pfu) LCMV (clone 13) via intravenous tail vein injection. On Day 5 post-infection, mice were treated with Saline or ONP-302 (1 mg/mouse) via intravenous tail vein injections on 5 consecutive days (Days 5-9). Mice were followed daily for weight loss. On Day 12 p.i mice were sacrificed; their spleens and blood were harvested. FIG. 2B-FIG. 2F show flow cytometric data of the splenocytes and leukocytes that were assayed. (*=p<0.05; **=p<0.005; ***=p<0.0005; ****=p<0.00005).

FIG. 3 shows the effect of ONP-302 on immune cells and viral titers in the spleen of mice infected with LCMV. C57BL/6 mice were infected with 2×10⁶ plaque forming units (pfu) LCMV (clone 13) via intravenous tail vein injection. On Day 5 post-infection, mice were treated with Saline or ONP-302 (1 mg/mouse) via intravenous tail vein injections on 5 consecutive days (Days 5-9). A subset of mice from each group were sacrificed on Days 12 and 35 p.i. Spleens and blood were harvested from mice. Splenocytes and leukocytes in blood were assayed by flow cytometry. LCMV viral titers in spleen were determined by plaque assay. (*=p<0.05; **=p<0.005; ***=p<0.0005; ****=p<0.00005).

FIG. 4A shows the effect of ONP-302 on lung function in aged mice infected with H1N1 influenza infection. Female C57BL/6 mice were anaesthetized and intranasally infected with 600 pfu H1N1 influenza virus. Mice were treated with Saline or ONP-302 beginning Day 3 p.i. Treatments were administered once daily for 5 consecutive days (Days 3-7). FIG. 4B shows lung function as assessed by daily measurements of oxygen saturation in blood using a pulse oximeter (MouseStat Jr.) (n=≥22). Mice were sacrificed on Day 9 p.i and the levels of monocytes (CD45+/CD11b+), inflammatory monocytes (CD45+/CD11b+/Ly6C+), and neutrophils (CD45+/Ly6G+) were assayed from bronchoalveolar lavage (BAL). FIG. 4C shows spleen data by flow cytometry. (n≥9). FIG. 4D shows levels of inflammatory protein. FIG. 4E shows MPO, FIG. 4E shows IL-6, and FIG. 4F shows CXCL-5 examined in BAL on Day 9 p.i using ELISA. (n >12). FIG. 4G shows lung damage assessed from the assay of damage marker Albumin from BAL. FIG. 411 shows lung tissues collected from mice on Day 9 p.i and subjected to histopathological analyses to assess levels of immune infiltration. (*=p<0.05; **=p<0.005; ***=p<0.0005; ****=p<0.00005).

FIG. 5A shows the effect of negatively charged particles ONP-302 at inhibiting pro-inflammatory cytokine production from human PBMCs stimulated with LPS ex vivo. Freshly isolated human PBMCs were cultured with indicated concentrations of ONP-302 for 30 minutes followed by co-incubation with 0.1 ng/mL LPS for 24 hours. Cell culture supernatants were harvested at 6, 12, and 24 hours after addition of LPS and the levels of IL-113 was determined. FIG. 5B shows levels of MCP-1. FIG. 5C shows levels of TNF-αas determined by ELISA.

FIG. 6 shows the effect of negatively charged particles ONP-302 on pro-inflammatory IL-6 production by monocytes stimulated with heat-killed bacteria (HK bacteria) (Staphylococcus aureus) in vitro. Mono-Mac-06 cells were co-incubated with 100 μg/mL CNP-301 and heat-killed bacteria (HK bacteria) (Staphylococcus aureus) for 24 hours. Unstimulated cells and saline were used as a negative control. 24-hours after incubation, the cell culture supernatant was harvested, and levels of IL-6 were assayed by ELISA.

### DETAILED DESCRIPTION

Cytokine Storm Syndrome (CSS) and Acute Respiratory Distress Syndrome (ARDS) are severe clinical conditions driven by a cascade of inflammatory events leading to overwhelming systemic inflammation, multiorgan dysfunction, and even death if untreated. CSS and ARDS are driven by pathological hyperinflammation due to the dysregulated activation and expansion of pro-inflammatory myeloid-derived cells (e.g monocytes, neutrophils, and macrophages) and production of excessive pro-inflammatory mediators (e.g cytokines, chemokines, and other proteins) leading to unchecked feedforward immune activation and amplification. This dysregulated inflammatory immune response gives rise to systemic inflammation resulting in multi-organ dysfunction and even death.

The present disclosure relates to negatively charged particles and compositions comprising the negatively charged particles as described herein for treating CSS and ARDS. The negatively charged particles of the present disclosure can ameliorate or alleviate the symptoms of CSS and ARDS. The negatively charged particles of the present disclosure are taken up preferentially by pro-inflammatory myeloid derived cells (e.g monocytes, neutrophils, and macrophages) which play a key role in the pathogenesis of CSS and ARDS. Particle uptake results in the sequestration of these cells in the liver and spleen in a non-inflammatory manner. As a result, fewer pro-inflammatory myeloid-derived cells are available for participation in the positive feedback loop driving pathological hyperinflammation during CSS and ARDS leading to resolution of inflammation. Preferentially targeting of pro-inflammatory myeloid derived cells leaves other immune regulatory and beneficial tissue repair functions intact ensuring resolution of pathologic inflammation without broad immune suppression and leading to improved recovery.

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in

the art, the following definitions are set forth to facilitate explanation of the presently disclosed subject matter.

It is noted here that as used in this specification and the appended claims, the singular forms “a,” “an,” and “the” include plural reference unless the context clearly dictates otherwise.

“Particle” as used herein refers to any non-tissue derived composition of matter, it may be a sphere or sphere-like entity, bead, or liposome. The term “particle”, the term “immune modifying particle”, and the term “bead” may be used interchangeably depending on the context. Additionally, the term “particle” may be used to encompass beads and spheres.

“Negatively charged particle” as used herein refers to particles which possess a net surface charge (also referred to herein as a zeta potential) that is less than zero. Zeta potential is the charge that develops at the interface between a solid surface and its liquid medium. A “Negative zeta potential” refers to a particle having a net surface charge of the particle that is less than zero, as represented in milliVolts (mV) and measured by an instrument known in the field to calculate zeta potential, e.g., a NanoBrook ZetaPlus zeta potential analyzer or Malvern Zetasizer. In embodiments, the negative zeta potential may be provided by anionic groups that are present on the surface of the particle.

In embodiments, the “negatively charged particle” may be a particle whose surface has been functionalized to provide a negative charge (referred to herein as a “surface-functionalized particle (SFP)”). In embodiments, surface functionalization occurs by the introduction of one or more functional groups to a surface of a particle. In embodiments, the negative charge may be provided by carboxylation (i.e., addition of one or more carboxyl groups to the particle surface) or addition of other anionic groups (groups bearing a negative charge in physiological pH), such as but not limited to sulfonic acid or phosphoric acid. In embodiments, the functional groups may be chemically conjugated to the surface of a particle, components of an overlayer disposed over the surface of a core (e.g., a bead), or a component of the material making up the particle and a sufficient amount of the functional groups are presented on the particle surface in order to provide the zeta potentials described herein. For example, acid-end capped PLGA polymers comprise carboxyl groups, and these carboxyl groups may be presented on the surface of the particle to provide a negatively charged particle having the zeta potentials described herein. In particular embodiments, the negatively charged particles comprise carboxyl groups on the particles' surface. Methods of making negatively charged particles are described in, for example, Froimowicz et al., Curr Org. Chem 17:900-912, 2013, or US 2020/0093753, each of which is incorporated by reference in its entirety for all purposes. In embodiments, it is contemplated that the negatively charged particles that are free of therapeutic agents, e.g., free from attached peptide or antigenic moieties or other bioactive agents. In some embodiments, the negatively charged particle may be further modified by the addition of targeting agents such as polypeptides, antibodies, nucleic acids, lipids, small-molecules, carbohydrates, and surfactants. While such further modifications are contemplated by this disclosure, the negatively charged particles described herein are able to treat ARDS or CSS without such modifications.

The term “subject” as used herein refers to a human or non-human animal, including a mammal or a primate, that is administered a particle as described herein. Subjects can include animals such as dogs, cats, rats, mice, rabbits, horses, pigs, sheep, cattle, and humans and other primates.

The term “therapeutic agent” refers to a moiety that is able to ameliorate or lessen one or more symptoms or signs of the disease or disorder being treated when administered at a therapeutically effective amount. Non-limiting examples of therapeutic agents include other therapeutics, including peptides, proteins, or small molecule therapeutic agents. For avoidance of doubt, the negatively charged particles of the disclosure, themselves, are therapeutically active and thus are therapeutic agent agents, and can treat the conditions described herein in the absence of additional, conventional therapeutic agents, such as peptides, proteins, or small molecule therapeutic agents.

The term “therapeutically effective amount” is used herein to indicate the amount of target- specific composition of the disclosure that is effective to ameliorate or lessen one or more symptoms or signs of the disease or disorder being treated.

The terms “treat”, “treated”, “treating” and “treatment”, as used with respect to methods herein refer to eliminating, reducing, suppressing or ameliorating, either temporarily or permanently, either partially or completely, one or more clinical symptom, manifestation or progression of an event, disease or condition. Such treating need not be absolute to be useful.

The following description includes information that may be useful in understanding the present disclosure. It is not an admission that any of the information provided herein is prior art or relevant to the presently claimed disclosures, or that any publication specifically or implicitly referenced is prior art.

Negatively charged Particles

The present disclosure relates to negatively charged particles and compositions comprising negatively charged particles. The present disclosure also relates to the use of the negatively charged particles for treating or ameliorating various diseases or conditions, including ARDS and/or CSS. The negatively charged particle of the present disclosure exhibits immunomodulatory properties. To be clear, the negatively charged particle of the present disclosure is a therapeutically active agent and is able to treat ARDS and/or CSS as the sole active agent.

Negatively charged particles can be formed from a wide range of materials. In embodiments, the particle is composed of a material suitable for biological use. In embodiments, the particle is composed of a pharmaceutically acceptable material. In embodiments, the particle comprises polymers, copolymers, dendrimers, diamond nanoparticle, polystyrene nanoparticles or metals. For example, particles may be composed of diamond, glass, silica, polyesters of hydroxy carboxylic acids, polyanhydrides of dicarboxylic acids, or copolymers of hydroxy carboxylic acids and dicarboxylic acids and biocompatible metals. In embodiments, the particles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, particles can be quantum dots, or composed of quantum dots, such as quantum dot polystyrene particles (Joumaa et al. (2006) Langmuir 22: 1810-6). Particles including mixtures of ester and anhydride bonds (e.g., copolymers of glycolic and sebacic acid) may also be employed. For example, particles may comprise materials including polyglycolic acid (PGA), polylactic acid (PLA), polysebacic acid polymers (PSA), poly(lactic-co-glycolic) acid copolymers (PLGA), [rho] oly(lactic-co-sebacic) acid copolymers (PL SA), poly(glycolic-co-sebacic) acid copolymers (PGSA), polypropylene sulfide polymers, poly(caprolactone) (PLC), chitosan, polysaccharide, sugar, hyaluronic acid, one or more lipids, a liposome, polyethylene glycol (PEG), cyclodextrin, etc.

Biocompatible, biodegradable polymers may also be used to form the negatively charged particles, including but not limited to polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in with any of the aforementioned materials. In embodiments, the particles include polymers of aspartic acid or glutamic acid, such as poly(aspartic acid), poly(gamma glutamic acid), or poly(glutamic acid). Biodegradable materials suitable for the present disclosure include PLA, PGA, polypropylene sulfide, and PLGA polymers, as well as metals such as iron (Fe), zinc (Zn), cadmium (Cd), gold (Au) or silver (Ag). Biocompatible but non-biodegradable materials may also be used in the particles described herein. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed. “Biodegradable” as used herein refers to a particle comprising a polymer that may undergo degradation, for example, by a result of functional groups reacting with the water in the solution. The term “degradation” as used herein refers to becoming soluble, either by reduction of molecular weight or by conversion of hydrophobic groups to hydrophilic groups. Biodegradable particles do not persist for long times in the body, and the time for complete degradation can be controlled.

In embodiments, the negatively charged particles of the disclosure are biodegradable within the body of a mammal. In embodiments, the particles of the disclosure are biodegradable within a human body. In embodiments, the particles of the disclosure undergo hydrolysis in the presence of water to produce safe byproducts. In embodiments, the particles of the disclosure undergo hydrolysis in vivo to produce safe byproducts.

In embodiments, the particle comprises one or more selected from: PGA, PLG, PLA, polystyrene, PLGA, PEG, chitosan, a lipid, sugar, hyaluronic acid, PCL, diamond, Fe, Zn, Cd, Au, or Ag.

In embodiments, the particle comprises PGA, PLA, polystyrene, or PLGA. In embodiments, the particle comprises PGA, PLA, or PLGA.

In particular embodiments, the particle comprises PLGA. PLGAs are safe and inherently biodegradable within the human body. PLGAs can undergo hydrolysis of ester linkages in the presence of water to produce lactic acids and glycolic acids, which are both safe at the contemplated dosage amounts of the particle as disclosed herein.

In embodiments, the negatively charged particle is a co-polymer having a molar ratio of two monomers in a range from about 99:1 to about 1:99, e.g., about 99:1, about 95:5, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, about 5:95, and about 1:99, including all values and ranges that lie in between these values. In embodiments, the particle is a co-polymer having a molar ratio of two monomers in a range from about 50:50 to about 99:1, from about 60:40 to about 95:5, from about 70:30 to about 90:10, or any values therein or any subranges therein.

In embodiments, the particle comprises PLGA having a molar ratio of polylactic acid:polyglycolic acid in a range from about 99:1 to about 1:99, including about 99:1, about 95:5, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, about 5:95, about 1:99, and all values and ranges that lie in between these values. In embodiments, the particle is a PLGA (a co-polymer of PLG and PLA) having a molar ratio of polyglycolic acid:polylactic acid ranging from about 10:90 to about 90:10, including from about 50:50 to about 90:10, from about 50:50 to about 80:20, from about 10:90 to about 50:50, from about 20:80 to about 50:50, or any values therein or any subranges therein. In particular embodiments, the particle comprises 50:50 polylactic acid:polyglycolic acid. In embodiments, the particle is a PLGA having a molar ratio of 50:50 polylactic acid:polyglycolic acid.

The particles of the disclosure can be manufactured by any means known in the art. Exemplary methods of manufacturing particles include, but are not limited to, microemulsion polymerization, interfacial polymerization, precipitation polymerization, emulsion evaporation, emulsion diffusion, solvent displacement, and salting out (Astete and Sabliov, J. Biomater. Sci. Polymer Edn., 17:247-289(2006)). Methods of making particles contemplated herein are disclosed in U.S. Pat. No. 9,616,113 and International Patent Publication WO/2017/143346. See also, US 2015/0010631 and US 2015/0174155, which are hereby incorporated by reference in their entireties.

Manipulation of the manufacturing process for PLGA particles can control particle properties (e.g. size, size distribution, zeta potential, morphology, hydrophobicity/hydrophilicity, polypeptide entrapment, etc). The size of the particle is influenced by a number of factors including, but not limited to, the concentration of polymer, e.g., PLGA, the solvent used in the manufacture of the particle, the nature of the organic phase, the surfactants used in manufacturing, the viscosity of the continuous and discontinuous phase, the nature of the solvent used, the temperature of the water used, sonication, evaporation rate, additives, shear stress, sterilization, and the nature of any encapsulated antigen or polypeptide.

It is contemplated that the particle may further comprise a surfactant. The surfactant can be anionic, cationic, or nonionic. The surfactant can be hydrophobic or hydrophilic. Surfactants in the poloxamer and poloaxamines family are commonly used in particle synthesis. Surfactants that may be used, include, but are not limited to polyvinyl alcohol (PVA), polyacrylic acid, PEG, Tween-80, gelatin, dextran, pluronic L-63, methylcellulose, lecithin, DMAB, PEMA, or combinations thereof. Additionally, biodegradable and biocompatible surfactants including, but not limited to, vitamin E TPGS (D-α-tocopheryl polyethylene glycol 1000 succinate) and polymers of amino acids (e.g lysine, arginine, aspartic acid, glutamic acid, serine, threonine, tyrosine and cysteine, or their enantiomers). In embodiments, process surfactants are selected from polyvinyl alcohol or polyacrylic acid, or combinations thereof. In certain embodiments, two surfactants are used. For example, if the particle is produced by a double emulsion method, the two surfactants can include a hydrophobic surfactant for the first emulsion, and a hydrophobic surfactant for the second emulsion. In embodiments, the particles are manufactured by nanoprecipitation, co-precipitation, inert gas condensation, sputtering, microemulsion, sol-gel method, layer-by-layer technique or ionic gelation method. Several methods for manufacturing nanoparticles have been described in the literature and are incorporated herein by reference ^{9,10}.

In embodiments, the particle of the disclosure has a negative zeta potential. In some embodiments, the zeta potential of the particle is in the range of about −100 mV to about −1 mV, including all values and ranges that lie in between these values. In embodiments, the zeta potential of the particle is in the range of about −80 mV to about −30 mV, including all values and ranges that lie in between these values. In embodiments, the zeta potential of the particle is from about −100 mV to about −40 mV, from about −80 mV to about −30 mV, from about −75 mV to about −40 mV, from about −70 mV to about −30 mV, from about −60 mV to about −45 mV, from about −60 mV to about −35 mV, from about −50 mV to about −40 mV, from about −55 mV to about −30 mV, from about −50 mV to about −35 mV, including all values and ranges that lie in between these values. In various embodiments, the zeta potential is about −30 mV, −35 mV, −40 mV, −45 mV, −50 mV, −55 mV, −60 mV, −65 mV, −70 mV, −75 mV −80 mV, −85 mV, −90 mV, −95 mV or −100 mV, including all values and subranges that lie between these values.

In embodiments, the particle has an average or mean diameter in the range of about 0.05 μm to about 15 μm, including all values and subranges that lie between these values. In embodiments, the particle has an average or mean diameter in the range of about 0.1 μm to about 10 μm, including all values and subranges that lie between these values. In embodiments, the particle has an average or mean diameter in the range of about 0.2 μm and about 2 μm, including all values and subranges that lie between these values. In embodiments, the particle has an average or mean diameter in the range of about 0.3 μm to about 5 μm, including all values and subranges that lie between these values. In embodiments, the particle has an average or mean diameter in the range of about 0.3 μm to about 3 μm, including all values and subranges that lie between these values. In embodiments, the particle has an average or mean diameter in the range of about 0.3 μm to about 1 μm, including all values and subranges that lie between these values. In embodiments, the particle has an average or mean diameter in the range of about 0.3 μm to about 0.8 μm, including all values and subranges that lie between these values. In embodiments, the particle has an average or mean diameter in the range of about 0.5 μm to about 1 μm, including all values and subranges that lie between these values. In embodiments, the particle has an average or mean diameter in the range of about 100 nm to about 1500 nm, about 200 nm to about 2000 nm, about 100 nm to about 1000 nm, about 300 nm to about 1000 nm, about 300 nm to about 900 nm, about 350 nm to about 850 nm, about 350 nm to about 850 nm, about 350 nm to about 750 nm, about 375 nm to about 825 nm, about 400 nm to about 800 nm, or about 200 nm to about 700 nm, including all values and subranges that lie between these values. In embodiments, the particle has an average or mean diameter of about 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm, including all values and subranges that lie between these values. In embodiments, the particle has an average or mean diameter in the range of about 300 nm to about 800 nm.

In embodiments, the polymer (e.g., PLGA) used to form the particle has a molecular weight ranging from about 500 to about 1,000,000 Da, e.g., 500 Da, 600 Da, 700 Da, 800 Da, 900 Da, 1,000 Da, 2,000 Da, 3,000 Da, 4,000 Da, 5,000 Da, 6,000 Da, 7,000 Da, 8,000 Da, 9,000 Da, 10,000 Da, 11,000 Da, 12,000 Da, 13,000 Da, 14,000 Da, 15,000 Da, 16,000 Da, 17,000 Da, 18,000 Da, 19,000 Da, 20,000 Da, 21,000 Da, 22,000 Da, 23,000 Da, 24,000 Da, 25,000 Da, 26,000 Da, 27,000 Da, 28,000 Da, 29,000 Da, 30,000 Da, 31,000 Da, 32,000 Da, 33,000 Da, 34,000 Da, 35,000 Da, 36,000 Da, 37,000 Da, 38,000 Da, 39,000 Da, 40,000 Da, 41,000 Da, 42,000 Da, 43,000 Da, 44,000 Da, 45,000 Da, 46,000 Da, 47,000 Da, 48,000 Da, 49,000 Da, 50,000 Da, 51,000 Da, 52,000 Da, 53,000 Da, 54,000 Da, 55,000 Da, 56,000 Da, 57,000 Da, 58,000 Da, 59,000 Da, 60,000 Da, 61,000 Da, 62,000 Da, 63,000 Da, 64,000 Da, 65,000 Da, 66,000 Da, 67,000 Da, 68,000 Da, 69,000 Da, 70,000 Da, 71,000 Da, 72,000 Da, 73,000 Da, 74,000 Da, 75,000 Da, 76,000 Da, 77,000 Da, 78,000 Da, 79,000 Da, 80,000 Da, 81,000 Da, 82,000 Da, 83,000 Da, 84,000 Da, 85,000 Da, 86,000 Da, 87,000 Da, 88,000 Da, 89,000 Da, 90,000 Da, 91,000 Da, 92,000 Da, 93,000 Da, 94,000 Da, 95,000 Da, 96,000 Da, 97,000 Da, 98,000 Da, 99,000 Da, or 100,000 Da, including all values and subranges that lie between these values.

In embodiments, the particles comprise (i) a biodegradable polymer (e.g., PGA, PLA, or PLGA), (ii) a zeta potential ranging from −100 mV to −30 mV (e.g., −100 mV, −90 mV, −80 mV, −70 mV, −60 mV, −50 mV, −40 mV, −30 mV, including all values and subranges that lie between these value), and (iii) an average particle diameter ranging from about 0.3 μm to about 5 μm (e.g., 0.3 μm, 0.4 μm, 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 2.3 μm, 2.4 μm, 2.5 μm, 2.6 μm, 2.7 μm, 2.8 μm, 2.9 μm, 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, or about 5.0 μm)

The size of the particle can be affected by the polymer concentration. Generally, larger particles are formed from higher polymer concentrations. For example, an increase in PLGA concentration from 1% to 4% (w/v) can increase mean particle size from about 205 nm to about 290 nm when the solvent propylene carbonate is used. Alternatively, in ethyl acetate and 5% Pluronic F-127, an increase in PLGA concentration from 1% to 5% (w/v) increases the mean particle size from 120 nm to 230 nm.

The viscosity of the continuous and discontinuous phase is also an important parameter that affects the diffusion process, a key step in forming smaller particles. The size of the particles increases with an increase in viscosity of the dispersed phase, whereas the size of the particles decreases with a more viscous continuous phase. In general, the lower the phase ratio of organic to aqueous solvent, the smaller the particle size.

Homogenizer speed and agitation also affect particle size. In general, higher speeds and agitation cause a decrease in particle size, although there is a point where further increases in speed and agitation no longer decrease particle size. There is a favorable impact in the size reduction when the emulsion is homogenized with a high-pressure homogenizer compared with just high stirring. For example, at a phase ration of 20% in 5% PVA, the mean particle size with stirring is 288 nm and the mean particle size with homogenization (high pressure of 300 bars) is 231 nm.

Particle size reduction can also be achieved by varying the temperature of the water added to improve the diffusion of the solvent. In general, the mean particle size decreases with an increase in water temperature.

The PLGA molecular mass can also impact the final mean particle size. In general, the higher the molecular mass, the higher the mean particle size. For example, as the composition and molecular mass of PLGA varies (e.g. 12 to 48 kDa for 50:50 PLGA; 12 to 98 kDa for 75:25 PLGA), the mean particle size varies (about 102 nm-154 nm; about 132 nm to 152 nm respectively). Even when particles are the same molecular mass, their composition can affect average particle size; for example, particles with a 50:50 ratio generally form particles smaller than those with a 75:25 ratio. The end groups on the polymer also affects particle size. For example, particles prepared with ester end-groups form particles with an average size of 740 nm (PI=0.394) compared with the mean size for the acid PLGA end-group is 240 nm (PI=0.225).

The solvent used can also affect particle size. In general, solvents that reduce the surface tension of the solution reduce particle size. During the preparation of the negatively charged particles, the organic solvent can be removed by evaporation in a vacuum to avoid polymer and polypeptide damage and to promote final particle size reduction. In general, evaporation of the organic solvent under vacuum is more efficient in forming smaller particles. For example, evaporation in vacuum produces a mean particle size around 30% smaller than the mean particle size produced under a normal rate of evaporation. Organic solvents that can be used in the production of the particles of the disclosure include, but are not limited to, ethyl acetate, methyl ethyl ketone, propylene carbonate, and benzyl alcohol. Other solvents that can be used in the production of the particles of the invention include, but are not limited to, acetone, tetrahydrofuran (THF), chloroform, and members of the chlorinate family, methyl chloride.

In embodiments, negative charge of the particle is achieved by the presence of carboxyl groups on the surface of the particle. In some embodiments, one or more carboxyl groups are conjugated on the particle's surface. Carboxylation can produce a negative charge on an otherwise neutral particle, or it can increase the negative charge of negatively charged particle. In embodiments, carboxylation of the particles can be achieved using any compound which adds carboxyl groups, including, but not limited to, poly(ethylene-maleic anhydride) (PEMA), poly acrylic acid (PAA), hyaluronic acid, poly amino acids. Without being bound by theory, carboxylation produces negatively charged surface, and this negative charge elicits therapeutic responses by instigating the uptake of the negatively charged particles by phagocytes and monocytes including macrophage receptor with collagenous (MARCO) structure monocytes. Without being bound by theory, it is believed that the negatively charged particles of the disclosure can bind to scavenger receptors expressed on monocytes and macrophages. It is believed that the negatively charged particles of the present disclosure can suppress the recruitment of pro-inflammatory monocytes to the lungs while leaving other regulatory immune process largely intact. In embodiments, the negatively charged particles, taken up by the pro- inflammatory monocytes and neutrophils, can redirect the monocytes and the neutrophils to the spleen and the liver where the pro-inflammatory monocytes and neutrophils can be sequestered and/or undergo apoptosis. This redirection of the monocytes and the neutrophils is believed to prevent the monocytes from releasing large quantities of pro-inflammatory proteins to the affected area, such as the lungs which can result in acute respiratory distress syndrome (ARDS) and/or organs such as liver, kidney, and CNS leading to multi-organ dysfunction and mortality associated with ARDS and CSS. In embodiments, the uptake of the negatively charged particles is by pro-inflammatory monocytes, neutrophils, and macrophages.

In models of acute hyperinflammation, including viral infections (e.g WNV), inflammatory monocytes were found to take up significantly more negatively charged particles than any other cell type. Treatment with negatively charged particles during acute inflammation led to sequestration of inflammatory monocytes in liver and spleens. For example, spleens from WNV-infected mice treated with FITC (fluorescein isothiocyanate) labeled negatively charged particles had significantly more inflammatory monocytes than those treated with neutral particles (not negatively charged) or vehicle control, closely corresponding to a decrease in circulating inflammatory monocytes in the peripheral blood in these WNV-infected mice. As a result, fewer inflammatory monocytes infiltrated into the brain resulting in resolution of inflammation. Further, Ly6Chi monocytes were sorted from the bone marrow of WNV-infected mice on D6 p.i., and labelled with PKH26, transferred i.v. into mock- or WNV-infected recipients on D6 p.i, which was followed immediately by injection with negatively charged particles, neutral particles or vehicle only. Migration of PKH26-labelled cells into the spleen was observed in mock- and WNV-infected mice, however, negatively charged particle treatment resulted in significantly more Ly6Chi monocyte accumulation in the spleens of WNV-infected mice. The data from these studies suggest that infused negatively charged particles are taken up by inflammatory monocytes, which are diverted to the spleen, resulting in reduced inflammatory monocyte numbers in the blood for migration into sites of inflammation. See U.S. Pat. No. 9,913,883, and Getts et al. (2014 Sci. Trans. Med) which are hereby incorporated by reference in its entirety for all purposes.

In another study, MARCO was found to be up-regulated on Ly6Chi/CD11b+/CD11c− ΦIM isolated from the spleen of WNV-infected but not mock-infected mice. The negatively charged particle treatment infusion in WT mice induced with peritoneal inflammation using thioglycollate resulted in the reduction of Ly6Chi/CD11b+ ΦIM in the peritoneum. However, Ly6Chi/CD11lb macrophages were not reduced in MARCO−/− (MARCO-deficient) mice also induced with thioglycollate, directly pointing for a role for MARCO in the uptake and efficacy of negatively charged particles. Interestingly, negatively charged particles significantly increased the numbers of apoptosis markers, annexin V and caspase-3 positive inflammatory monocytes, in the spleens of WT mice but not MARCO−/− mice 2 hours after infusion of negatively charged particles. The data suggest that negatively charged particles are likely to be taken up through the MARCO scavenger receptor, which may mediate downstream signaling pathways that result in inflammatory monocyte migration, accumulation and subsequent apoptosis in the spleen. See U.S. Pat. No. 9,913,883, which is hereby incorporated by reference in its entirety for all purposes.

Accordingly, these studies indicate that the negatively charged particles described herein are taken up by inflammatory myeloid-derived cells (e.g monocytes) and would be useful for treating patients suffering from CSS and/or ARDS.

In some embodiments, negative charging of subject particles is achieved by the addition of targeting agents. In some embodiments, the targeting agent comprises peptides, polypeptides, antibodies, carbohydrates, nucleic acids, lipids, small molecules, and surfactants.

In embodiments, the negatively charged particles are targeted preferentially to monocytes, neutrophils, macrophages, T-cells, B-cells, NK cells, NK T-cells, fibroblasts, endothelial cells, adipocytes, pericytes, endothelium, vasculature, lymphatic vessels, mesenchymal stromal cells, mesenchymal stem cells, and/or extracellular matrix.

In embodiments, the negatively charged particles targets (taken up by) monocytes, neutrophils, and macrophages. In embodiments, the negatively charged particles target pro-inflammatory monocytes, neutrophils, and macrophages. In embodiments, the negatively charged particles target pro-inflammatory monocytes, neutrophils, and macrophages that are recruited by immune signaling during CSS and ARDS triggered by viral infections, bacterial infections, tissue injury, pathogens, immune-directed therapies (e.g CAR-Ts, antibodies, and cytokines), autoimmune and rheumatic conditions (e.g arthritis and lupus), macrophage activation syndrome (MAS), reactive hemophagocytic syndrome, secondary hemophagocytic lymphohistiocytosis (sHLH), opportunistic infections, pulmonary contusions, inhalation injury from chemicals, particulates, or other irritants, aspirations of gastric contents, near drowning. Examples of ARDS resulting from indirect lung injury include hemorrhagic shock, pancreatitis, major burn injury, drug overdose, transfusion of blood products, cardiopulmonary bypass, sepsis, and reperfusion injury. In embodiments, the negatively charged particles target pro-inflammatory monocytes, neutrophils, and macrophages activated and contributing to pathologic hyperinflammation in response to a respiratory infection.

In embodiments, the negatively charged particles do not comprise another therapeutically active agent (e.g., the only therapeutically active agent is the negatively charged particles themselves). In embodiments, the negatively charged particles are free (i.e., do not include) from another therapeutically active agent.

### Compositions Comprising Negatively Charged Particles

To administer particles as described herein to human or other mammals, the particle may

be formulated in a composition comprising one or more pharmaceutically acceptable carriers. The phrase “pharmaceutically or pharmacologically acceptable” refer to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like. In embodiments, the composition comprising the particles as disclosed herein is a sterile composition.

Pharmaceutical compositions of the present disclosure comprising the particle disclosed herein may contain pharmaceutically acceptable carriers or additives depending on the route of administration. The pharmaceutical composition can be suitable for administration orally, nasally, transdermally, pulmonary, inhalationally, buccally, sublingually, intraperintoneally, subcutaneously, intramuscularly, intravenously, rectally, intrapleurally, intrathecally, intraportally, or parenterally. In embodiments, the pharmaceutical composition comprising the negatively charged particle disclosed herein is for intravenous administration. In embodiments, the pharmaceutical composition is a solution for injection. In embodiments, the pharmaceutical composition is a ready-to-use formulation for intravenous administration. In embodiments, the pharmaceutical composition is a solid formulation. In embodiments, the pharmaceutical composition is a lyophilized composition that is reconstituted when used.

In embodiments, the pharmaceutically acceptable carrier or additive is selected from one or more of: a binder, lubricant, inert diluent, cryoprotectant, buffering agent, flavoring agent, preservative, disintegrant, or dispersing agent.

Non-limiting examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present disclosure. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers. A variety of aqueous carriers are suitable, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

Pharmaceutical compositions comprising the particle can be prepared for storage by mixing the particle having the desired degree of purity with optional physiologically acceptable carriers, excipients or stabilizers (see Remington's Pharmaceutical Sciences 16th edition, Osol, A. Ed. (1980)). In embodiments, the pharmaceutical composition comprising the particle is in the form of lyophilized formulations or aqueous solutions. Acceptable carriers, excipients, or stabilizers are nontoxic to recipients at the dosages and concentrations employed, and include buffers such as phosphate, citrate (e.g., sodium citrate dihydrate), succinate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; or metal complexes (e.g., Zn- protein complexes). In embodiments, sugars can be used as a cryoprotectant.

Preparations of the particles can be stabilized by lyophilization. The addition of a cryoprotectant such as trehalose, sucrose, and/or mannitol (e.g., D-mannitol), can decrease aggregation of the particles upon lyophilization. In embodiments, the lyophilized composition comprising the particle as disclosed herein also comprises one or more of cryoprotectant and buffering agent.

Any suitable lyophilization and reconstitution techniques can be employed. Lyophilized compositions comprising the particles can be reconstituted with sterile injection solutions for intravenous administration. In embodiments, the pharmaceutical composition comprises a lyophilized particle in sterile injection solution. In embodiments, the pharmaceutical composition comprises a lyophilized particle in sterile water for injection.

In embodiments, the composition of the present disclosure can be in a form of a kit. In embodiments, a kit comprises a solid composition comprising the negatively charged particles and a separate composition comprising solution suitable for injection. Solution suitable for injection is a sterile solution. In embodiments, the sterile solution for injection is selected from water, glucose solution, dextrose solution, sucrose solution, and saline. In embodiments, a kit comprises a lyophilized composition comprising the negatively charged particles and a separate composition comprising solution suitable for injection.

In any one of the kits as disclosed herein, the kit can further comprise syringes, filters, and/or instructions for use.

### Therapeutic Use of the Negatively Charged Particles

The present disclosure relates to use of the negatively charged particles as disclosed herein for treating cytokine storm syndrome (CSS) and acute respiratory distress syndrome (ARDS). In embodiments, the negatively charged particles are useful for treating the symptoms of CSS and ARDS. CSS and ARDS are critical clinical conditions driven by a cascade of inflammatory events leading to overwhelming systemic inflammation, multiorgan dysfunction, and even death. CSS and ARDS are driven by pathological hyperinflammation due to the dysregulated activation and expansion of pro-inflammatory myeloid-derived cells (e.g monocytes, neutrophils, and macrophages) and production of excessive pro-inflammatory mediators (e.g cytokines, chemokines, and other proteins) leading to unchecked feedforward immune activation and amplification. This dysregulated inflammatory immune response gives rise to systemic inflammation resulting in multi-organ dysfunction and even death.

In one aspect, it is hypothesized that the negatively charged particles are taken up by phagocytic cells (e.g monocytes, neutrophils, and macrophages) via scavenger mechanisms. Without bound to any theory, once the negatively charged particles are taken up by pro-inflammatory monocytes, macrophages, and neutrophils, the particles can redirect the cells to the spleen or the liver where they can be sequestered and/or undergo apoptosis. In embodiments, the particles of the present disclosure redirect the pro-inflammatory monocytes, neutrophils, and macrophages. In embodiments, the particles of the present disclosure redirect pro-inflammatory monocytes. In embodiments, the particles of the present disclosure redirect pro-inflammatory neutrophils. In embodiments, the particles of the present disclosure redirect the pro-inflammatory monocytes, neutrophils away from the lungs. In embodiments, the redirecting of the pro- inflammatory monocytes and neutrophils away from the site of inflammation (e.g lungs) can prevent or ameliorate the release of inflammatory proteins that can progress to CSS and ARDS. In embodiments, the redirecting of the pro- inflammatory monocytes, macrophages, and neutrophils away from the site of inflammation can prevent or ameliorate CSS that can progress to ARDS and vice versa. In embodiments, negatively charged particles of the present disclosure can suppress the recruitment of pro-inflammatory monocytes, neutrophils, and macrophages to sites of inflammation while leaving other regulatory immune process largely intact or unperturbed. In embodiments, negatively charged particles of the present disclosure do not cause broad immune suppression.

In embodiments, the particles of the present disclosure are taken up by monocytes, neutrophils, and macrophages. In embodiments, particle uptake results in the reprogramming of monocytes, neutrophils, and macrophages. In embodiments, particle uptake results in the reprogramming of monocytes, neutrophils, and macrophages from a pro-inflammatory type to an anti-inflammatory type. In embodiments, particle uptake results in reprogramming of monocytes and macrophages from the pro-inflammatory M1 type to the anti-inflammatory M2 type. In embodiments, particle uptake results in reprogramming of neutrophils from the inflammatory N1 type to the anti-inflammatory N2 type.

In any one of the methods as disclosed herein, the subject suffering from CSS and/or ARDS suffers from a viral infection, a bacterial infection, sepsis, cytokine release syndrome (CRS), severe inflammatory response syndrome (SIRS), hypercytokinemia, macrophage activation syndrome (MAS), systemic juvenile idiopathic arthritis—associated macrophage activation syndrome (systemic JIA-MAS), non-systemic JIA-MAS, NLRC4-MAS, systemic JIA, malignancy-associated hyperinflammation (MASH), reactive hemophagocytic syndrome, hemophagocytic lymphohistiocytosis (HLH), secondary hemophagocytic lymphohistiocytosis (sHLH), familial hemophagocytic lymphohistiocytosis (FHLH), Epstein-Barr virus-associated hemophagocytic lymphohistiocytosis (EBV-HLH), a traumatic injury, adult onset Still's disease, systemic lupus erythematosus, Kawasaki disease, or combinations thereof.

In any one of the methods disclosed herein, CSS and/or ARDS is the result of lung injury. In embodiments, the lung injury is direct lung injury or indirect lung injury. Non-limiting examples of CSS and/or ARDS resulting from direct lung injury include pneumonia due to bacterial, viral, fungal, or opportunistic infections; pulmonary contusions; traumatic injuries; inhalation injury from chemicals, particulates, or other irritants; aspirations of gastric contents; and near drowning. Non- limiting examples of CSS and/or ARDS resulting from indirect lung injury include hemorrhagic shock, pancreatitis, major burn injury, drug overdose, transfusion of blood products, cardiopulmonary bypass, sepsis, and reperfusion injury. In embodiments, the lung injury is a result of sepsis, pneumonia, viral infection, bacterial infection, fungal infection, opportunistic infection, pulmonary contusions, traumatic injuries, inhalation injury from chemicals, particulates, or other irritants, aspirations of gastric contents, near drowning, hemorrhagic shock, pancreatitis, major burn injury, drug overdose, transfusion of blood products, cardiopulmonary bypass, and/or reperfusion injury.

In any one of the methods disclosed herein, CSS and/or ARDS is the result of pneumonia, lung inflammation, bacterial infection, viral infection, fungal infection, opportunistic infection, sepsis, aspiration of gastric contents, traumatic injury, burn injury, pancreatitis, pulmonary contusion, hemorrhagic shock, near drowning, blood transfusion, inhalation injury, or combinations thereof.

In any one of the methods disclosed herein, CSS and/or ARDS is the result of a viral infection or bacterial infection. CSS and/or ARDS resulting from a viral or bacterial infection in a patient are not due to the infection itself but due to the pathological hyperinflammatory response to the infection, and therefore CSS or ARDS develops only in patients which develop a hyperinflammatory response to the infection. In cases of respiratory infections, CSS and/or ARDS can develop in a patient when the monocytes and associated immune cells overwhelm the lungs releasing large quantities of pro- inflammatory proteins. For example, SARS coronavirus-2 discovered in December 2019 has demonstrated greater risk of developing CSS and ARDS, especially with older patients with co-morbid conditions including diabetes, chronic cardiovascular disease, chronic pulmonary disease, chronic renal disease, cancer, and/or immunodeficiencies. In embodiments, CSS and/or ARDS is the result of viral infection.

In any one of the methods disclosed herein, ARDS is the result of a viral infection caused by a DNA virus, an RNA virus, and/or a retrovirus. In embodiments, the DNA virus is a single- stranded DNA (ssDNA) virus, or a double-stranded (dsDNA) virus, and the RNA virus is a double- stranded RNA virus, a single-stranded RNA (ssRNA) (+) virus, ssRNA (−) virus, or a circular ssRNA virus.

In embodiments, ssDNA virus is selected from Anellovirus, Circovirus, Genomovirus, or Parvovirus. In embodiments, the Anellovirus is Alphatorquevirus, Betatorquevirus, or Gammatorquevirus. In embodiments, the Circovirus is a Cyclovirus. In embodiments, the Genomovirus is Gemycircular virus, Gemykibivirus, or Gemyvongvirus. In embodiments, the Parvovirus is Erythovirus, Dependovirus, or Bocavirus. In embodiments, dsDNA virus is selected from Herpesvirus, Adenovirus, Papillomavirus, Polyomavirus, or Poxvirus. In embodiments, the Herpesvirus is Simplexvirus, V ericellovirus, V ytomegalovirus, Roseolovirus, Lymphocryptovirus, or Rhadinovirus. In embodiments, the Adenovirus is Mastadenovirus. In embodiments, the Papillomavirus is Alpha-apillomavirus, Beta-papillomavirus, Gamma- papillomavirus, Mupapillomavirus, or Nupapapillomavirus. In embodiments, the Polyomavirus is Alpha-polyomavirus, Beta-polyomavirus, Gamma-polyomavirus, or Delta-polyomavirus. In embodiments, the Poxyvirus is Molluscipoxivirus, Orthopoxivirus, or Parapoxivirus.

In embodiments, the retrovirus is Hepadnavirus, Orthohepadnavirus, Gammaretrovirus, Deltaretrovirus, Lentivirus, or Simlispurnavirus. In embodiments, the dsRNA virus is Picobirnavirus or Reovirus. In embodiments, the Reovirus is Voltivirus, Rotavirus, or Seadornavirus.

In embodiments, the ssRNA (+) virus is Coronavirus, Astrovirus, Calicivirus, Flavivirus, Hepevirus, Matonavirus, Picornavirus, or Togavirus. In embodiments, the Coronavirus is Alpha- coronavirus, Beta-coronavirus, or Torovirus. In embodiments, the Astrovirus is Mamastrovirus. In embodiments, the Calicivirus is Norovirus or Sapovirus. In embodiments, the Flavivirus is Hepacivirus or Pegivirus. In embodiments, the Hepevirus is Orthohepevirus. In embodiments, the Matonavirus is Rubivirus. In embodiments, the Picornavirus is Cardiovirus, Cosavirus, Enterovirus, Hepatovirus, Kobuvirus, Parechovirus, Rosavirus, or Salivirus. In embodiments, the Togavirus is Alphavirus. In embodiments, the ssRNA (−) virus is Filovirus, Paramyxovirus, Pneumovirus, Rhabdovirus, Arenavirus, Hentavirus, Nairovirus, Preibunyavirus, Phenuvirus, or Orthomyxovirus. In embodiments, the Filovirus is Ebola virus or Marburg virus. In embodiments, the Paramyxovirus is Henipavirus, Morbilivirus, Repirovirus, or Rubulavirus. In embodiments, the Pneumovirus is Metapneumovirus or Orthopneumovirus. In embodiments, the Rhabdovirus is Ledantevirus, Lyssavirus, or Vesiculovirus. In embodiments, the Arenavirus is Mammarenavirus. In embodiments, the Hantavirus is Orthohantavirus. In embodiments, the Nairovirus is Orthonairovirus. In embodiments, the Peribunyavirus is Orthobunyavirus. In embodiments, the Phenuvirus is Phlebovirus. In embodiments, the Orthomyxovirus is Alpha-influenza virus, Beta- influenza virus, Gamma-influenza virus, Quaranjavirus, or Thogotovirus.

In any one of the methods disclosed herein, CSS and/or ARDS is the result of a viral infection caused by a respiratory virus. In embodiments, the viral infection is caused by a virus is selected from Adenovirus, Adeno-associated virus, Aichi virus, Australian Bat Lyssavirus, BK polyomavirus, Banna virus, Barmah forest virus, Bunyavirus snowshoe hare, Cercopithecine herpes virus, Chandipura virus, Chikungunya virus, Cosavirus A, Cowpox virus, Coxsackievirus, Crimean- Congo hemorrhagic fever virus, Dengue virus, Dhori virus, Dugbe virus, Duvenhage virus, Eastern equine encephalitis virus, Echovirus, Encephalomyocarditis virus, Epstein-Barr virus, European Bat Lyssavirus, GB virus C/Hepatitis G virus, Hantaan virus, Hendra virus, Hepatitis A virus, Hepatitis B virus, Hepatitis C virus, Hepatitis delta virus, Human adenovirus, Human astrovirus, Human coronavirus, Human cytomegalovirus, Human enterovirus 68,70, Human herpesvirus 1, Human herpesvirus 2, Human herpesvirus 6, Human herpesvirus 7, Human Immunodeficiency Virus (HIV), Human papillomavirus 1, Human papillomavirus 2, Human papillomavirus 16,18, Human parainfluenza virus, Human parvovirus B19, Human respiratory syncytial virus, Human rhinovirus, Human SARS coronavirus, Human spumarterovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro visu, MERS coronavirus, Measles virus, Mengo encephalomyocarditis, Merkell cell polyomavirus, Mokola virus, Molluscum contagiosum virus, Monkeypox virus, Mumps virus, Murray valley encephalitis virus, New York virus, Nipah virus, Norwalk virus, O'Nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama, Salivirus A, Sandfly fever sicilian virus, Sapporo virus, SARS coronavirus-2, Semliki forest virus, Seoul virus, Simian foamy virus, Simian virus 5, Sindbis virus, Southampton virus, St. Louis encephalitis virus, Tick-borne Powassan virus, Torque teno virus, Toscana virus, Uukuniemi virus, Varicella-zoster virus, Variola virus, Venezuelan equine encephalitis virus, WU polyomavirus, Yaba Monkey tumor virus, Yaba-like disease virus, Yellow fever virus, or Zika virus. In embodiments, the viral infection is coronavirus. In embodiments, the viral infection is human coronavirus. In embodiments, the viral infection is human SARS coronavirus, or SARS coronavirus 2.

In any one of the methods disclosed herein, CSS and/or ARDS is the result of a bacterial infection. In embodiments, the bacterial infection is due to Staphylococcus, Streptococcus, Mycobacterium, Bacillus, Salmonella, Vibrio, Spirochete, Neisseria, Diplococcus, Pseudomonas, Clostridium, Treponaema, Spirillum, or combinations thereof.

In any one of the methods disclosed herein, the subject suffers from CRS and/or ARDS due to one or more immune-targeted therapies. In embodiments, the immune-targeted therapy is an antibody, a protein therapeutic, a peptide, a cytokine, an immune signaling modulator, an mRNA, an oncolytic virus, or a cell-based therapy.

In any one of the methods disclosed herein, the subject suffers from CRS and/or ARDS due to one or more antibody therapies. In embodiments, the antibody used in the antibody therapy is a monoclonal antibody, a polyclonal antibody, a bi-specific antibody, a tri-specific antibody, or a bi- specific T-cell engager (BiTE) antibody. In embodiments, the antibody used in the antibody therapy targets one or more of: CD2, CD3, CD20, CD27, CD28, CD30, CD4OL, CD137, OX-40, GITR, LIGHT, DR3, SLAM, ICOS, LILRB2, LILRB3, LILRB4, PD-1, PD-L1, CTLA-4, IL-12, or IL-15. In embodiments, the antibody targets receptor tyrosine kinase (RTK), EGFR, VEGF, VEGFR, PDGF, PDGFR, HER2/Neu, ER, PR, TGF-β1, TGF-β2, TGF-β3, SIRP-α, PD-1, PD-L1, CTLA-4, CD3, CD25, CD19, CD20, CD39, CD47, CD73, FAP, IL-1β, IL-2R, IL-12, IL-15, IL-15R, IL-23, IL-33, IL-2R, IL-4Rα, T-cells, B-cells, NK cells, macrophages, monocytes, and/or neutrophils.

In any one of the methods disclosed herein, the subject suffers from CRS and/or ARDS due to one or more cytokine therapies. In embodiments, the cytokine used in the cytokine therapy is selected from IFN-α, IFN-γ, IL-2, IL-10, IL-12, IL-15, IL-15/IL-15Rα, IL-18, IL-21, GM-CSF, or variants thereof.

In any one of the methods disclosed herein, the subject suffers from CRS and/or ARDS due to one or more immune signaling modulator therapies. In embodiments, the immune signaling modulator used in the immune signaling modulator therapy targets one or more of: IL-1R, IL-2Rα, IL-2Rβ, IL-2Rγ, IL-3Rα, CSF2RB, IL-4R, IL-5Rα, CSF2RB, IL-6Rα, gp130, IL-7Rα, IL-9R, IL-10Rα, IL-10Rβ, IL-12Rβ1, IL-12Rβ2, IL-13Rα1, IL-13Rα2, IL-15Rα, IL-21R, IL23R, IL-27Rα, IL- 31Rα, OSMR, CSF-1R, GM-CSF-R, cell-surface IL-15, IL-10Rα, IL-10Rβ, IL-20Rα, IL-20Rβ, IL-22Rα1, IL-22Rα2, IL-22Rβ, IL-28RA, TLR, JAK, BTK, TYK, SYK, MAPK, PI3K, NFκB, NFAT, STAT, or a kinase.

In embodiments, the subject suffers from CRS due to one or more cell-based therapies. In embodiments, the cell-based therapy comprises allogenic, autologous, or iPSC-derived cells. In embodiments, the cell-based therapy comprises one or more of: T-cells, NK-cells, red blood cells, stem cells, antigen-presenting cells, macrophages, or dendritic cells.

In any one of the methods disclosed herein, CSS and/or ARDS is the result of viral infections, bacterial infections, tissue injury, pathogens, immune-directed therapies (e.g CAR-Ts, antibodies, and cytokines), autoimmune and rheumatic conditions (e.g arthritis and lupus), macrophage activation syndrome (MAS), reactive hemophagocytic syndrome, secondary hemophagocytic lymphohistiocytosis (sHLH), opportunistic infections, pulmonary contusions, inhalation injury from chemicals, particulates, or other irritants, aspirations of gastric contents, near drowning. Examples of ARDS resulting from indirect lung injury include hemorrhagic shock, pancreatitis, major burn injury, drug overdose, transfusion of blood products, cardiopulmonary bypass, sepsis, and reperfusion injury. Following events triggering CSS and/or ARDS, a robust and heightened immune response can be elicited, involving activation of resident immune cells that produce pro-inflammatory cytokines and chemokines followed by the rapid influx of inflammatory monocytes and neutrophils to the site of the injury within about 24 to 48 hours of initial injury. The peripherally derived pro-inflammatory monocytes, neutrophils, and macrophages infiltrating at the site of injury respond to the local inflammatory milieu and further promote inflammation via production of pro-inflammatory cytokines (e.g., IL-1βIL-2, IL-6, IL-7, IL-8, IL-10, TNF-α, IFN-γ, IP-10, GM-CSF), chemokines (e.g., CCL-2, CXCL-1, CXCL-2, MIP-1β, MCP-1, and CXCL-5), oxidants (e.g., ROS), proteins (e.g., c-reactive protein), proteases (e.g MMPs and MPO), and neutrophil extracellular traps (NETs). A certain degree of inflammation is important for the resolution of an injury; however, excessive and prolonged inflammation can result in significant tissue/organ damage which can be life-threatening.

The present disclosure relates to reducing the accumulation of inflammatory mediators in the lungs, comprising administering the negatively charged particles as disclosed herein. In some embodiments, the inflammatory mediators are reduced by about 10%, 15%, 20%, 25%, 30%, 35%, 40%, 45%, 50%, 55%, 60%, 65%, 70%, 75%, 80%, 85%, 90%, 95%, 99%, or 100% compared to a patient with CSS and/or ARDS that does not receive treatment with the negatively charged particles disclosed herein. The present disclosure also relates to altering the levels of inflammatory mediators present systemically and/or at the sites of inflammation (e.g lungs) of a CSS and/or ARDS patient, comprising administering the negatively charged particles as disclosed herein.

The present disclosure also relates to reducing the levels of inflammatory mediators in circulation of a CSS and/or ARDS patient, comprising administering the negatively charged particles as disclosed herein. In some embodiments, the inflammatory mediators are reduced at least about 10%, 15%, 20%, 25%, 30%, 35%, 40%, 45%, 50%, 55%, 60%, 65%, 70%, 75%, 80%, 85%, 90%, 95%, 99%, or 100% compared to a patient with CSS and/or ARDS that does not receive treatment with the negatively charged particles disclosed herein. The present disclosure also relates to altering the levels of inflammatory mediators in circulation and/or site(s) of inflammation of a CSS and/or ARDS patient, comprising administering the negatively charged particles as disclosed herein. The present disclosure also relates to regulating the levels of inflammatory mediators in site(s) of inflammation and/or circulation of a CSS and/or ARDS patient, comprising administering the negatively charged particles as disclosed herein.

In embodiments, the inflammatory mediators include immune cells, proteases, oxidants, enzymes, eicosanoids, phospholipids, proteins, cytokines, chemokines, and metabolites. In embodiments, the inflammatory mediators are selected from one or more of: immune cells, cytokines, chemokines, oxidants, enzymes, proteins, or proteases. In embodiments, the inflammatory mediator damages distal cells or induces distal cell death. In embodiments, the inflammatory mediator damages alveolar type 2 epithelial cells or induces alveolar type 2 epithelial cells death. In embodiments, reducing the accumulation of the inflammatory mediator is necessary when the inflammatory mediator concentration is abnormally high. In embodiments, the method as disclosed herein reduces the concentration of the inflammatory mediator. In embodiments, the method as disclosed herein reduces the concentration of the inflammatory mediator to a normal level.

In embodiments, the immune cells are antigen-presenting cells (APCs), monocytes, neutrophils, macrophages, granulocytes, dendritic cells, T-cells, B-cells, and/or NK cells. In embodiments, the cytokine is selected from one or more of: IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-17, IL-18, IL-20, IL-21, IL-22, IL-23, IL-24, IL-25, IL-26, IL-27, IL-28, IL-29, IL-30, IL-31, IL-32, IL-33, IL-35, IL-36, IFN-α, IFN-β, IFN-γ, TNF-α, TGF-β, TGF-β, or TGF-β3. In embodiments, the chemokine is selected from one or more of: CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP- 1 α), CXCL4 (MIP-1β, CXCL5 (RANTES), CXCL6, CXCL7, CXCL8, CXCL9, CXCL10, CXCL11, CXCL12, CXCL13, CXCL14, CXCL15, CXCL16, or CXCL17. In embodiments, the protease is selected from one or more of: ADAM1, ADAM2, ADAM7, ADAMS, ADAMS, ADAM10, ADAM11, ADAM12, ADAM15, ADAM17, ADAM18, ADAM19, ADAAM20, ADAM21, ADAM22, ADAM23, ADAM28, ADAM29, ADAM30, ADAM33, MMP1, MMP2, MMP3, MMP7, MMP8, MMP 9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, MMP28, or Neutrophil elastase. In embodiments, the enzyme is selected from one or more of: cyclo-oxygenase-1 (COX-1), cyclo- oxygenase-2 (COX-2), 5-lipo-oxygenase (5-LOX), myeloperoxidase (MPO), and neutrophil elastase (NE). In embodiments, the protein is an apoptosis regulator. In embodiments, the apoptosis regulator is selected from one or more of: P53, Caspase 1, Caspase 2, Caspase 3, Caspase 4, Caspase 5, Caspase 6, Caspase 7, Caspase 8, Caspase 9, Caspase 10, Caspase 11, Caspase 12, Caspase 13, Caspase 14, BCL-2, BCL- XL, MCL-1, CED-9, A1, BFL1, BAX, BAK, DIVA, BCL-XS, BIK, BIM, BAD, BID, or EGL-1. In embodiments, the inflammatory mediator is neutrophil extracellular traps (NETs). In embodiments, the inflammatory mediator is cathepsin G. In embodiments, the inflammatory mediator is peptidyl arginine deaminase 4 (PADI-4). In embodiments, the inflammatory mediator is immunoglobins (Ig). In embodiments, the immunoglobulins are selected from one or more of: IgA, IgD, IgE, IgM, or variants thereof. A list of human metabolites that can be assayed from a biological sample can be found in the literature including in (Psychogios et al., 2011), (Wishart et al., HMDB: the Human Metabolome Database. Nucleic Acids Res. 2007 Jan; 35(Database issue):D521-6, 2007), and the Human Metabolome Database (HMDB) and are incorporated herein by reference.

In embodiments, the methods disclosed herein reduce the accumulation of cytokine and/or chemokine inflammatory mediators. In embodiments, the inflammatory mediator is a pro-inflammatory cytokine or chemokine. In embodiments, the pro-inflammatory cytokines and chemokines are selected from the group consisting IL-1β,IL-2, IL-6, IL-7, IL-8, IL-10, TNF-α, IFN-γ, IP-10, GM-CSF, CCL-2, CXCL-1, CXCL-2, MIP-1β, MCP-1, and CXCL-5. In embodiments, the pro-inflammatory cytokine is selected from IL-1β, IL-6, IL-8, IL-18, or TNF such as TNF-α. In embodiments, the inflammatory mediator is IL-1β, IL-6, TNF, MCP-1, thrombin, vascular endothelial growth factor (VEGF), and/or alarmin (damage-associated molecular patterns; DAMP). In embodiments, the inflammatory mediator is selected from one or BMP-15, CXCL16, CXCR3, IL-6, NOV/CCN3, glypican 3, IGFBP-4, IL-5, IL-5 Rα, IL-22 BP, leptin, MIP-1d, or orexin B. In embodiments, the inflammatory mediator is a chemokine. In embodiments, the chemokine is selected from CCL-2, CXCL-1, CXCL-2, and CXCL-5. In embodiments, the inflammatory mediator is an oxidant. In embodiments, the oxidant is a reactive oxygen species (ROS). In embodiments, the inflammatory mediator is a protein. In embodiments, the protein is a c-reactive protein. In embodiments, the inflammatory mediator is a protease. In embodiments, the inflammatory mediator is neutrophil extracellular traps (NET).

The levels of the inflammatory mediators in a patient can be measured in the patient's whole blood, serum, plasma, PBMCs, urine, cerebrospinal fluid (CSF), stool, a tissue biopsy, and/or a bone-marrow biopsy. In embodiments, the inflammatory mediators in patient's blood, serum, or plasma can be measured by antibody microarray. See Chen et al. Cell Biol Toxicol (2016) 32:169-184. The levels of the inflammatory mediators in a patient can also be measured in the patient's bronchoalveolar lavage (BAL). See Grazioli S. et al. (2019) PLoS ONE 14(11):e0225468.

The present disclosure also relates to altering the levels of cell-surface proteins on immune cells in a CSS and/or ARDS patient, comprising administering the negatively charged particles as disclosed herein. The present disclosure also relates to regulating the levels of cell-surface proteins on immune cells in an ARDS patient, comprising administering the negatively charged particles as disclosed herein. The present disclosure also relates to reducing the levels of cell- surface proteins on immune cells in a CSS and/or ARDS patient, comprising administering negatively charged particles as disclosed herein. In embodiments, the cell-surface proteins are reduced by about 10%, 15%, 20%, 25%, 30%, 35%, 40%, 45%, 50%, 55%, 60%, 65%, 70%, 75%, 80%, 85%, 90%, 95%, 99%, or 100% compared to a patient with CSS and/or ARDS that does not receive treatment with the negatively charged particles disclosed herein. In embodiments, the cell- surface protein is selected from one or more of: CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD3OL, CD31, CD32, CD32b, CD34, CD33, CD38, CD39, CD40, CD4O-L, CD41b, CD42a, CD42b,CD43, CD44, CD45, CD45RA, CD47, CD45RA, CD45RO, CD48, CD52, CD55, CD56, CD58, CD61, CD66b, CD69, CD70, CD72, CD79, CD68, CD84, CD86, CD93, CD94, CD95, CRACC, BLAME, BCMA, CD103, CD107, CD112, CD120a, CD120b, CD123, CD125, CD127, CD134, CD135, CD140a, CD141, CD154, CD155, CD160, CD161, CD163,CD172a, XCR1, CD203c, CD204, CD206, CD207 CD226, CD244, CD267, CD268, CD269, CD355, CD358, CRTH2, NKG2A, NKG2B, NKG2C,NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβεRI, MHC-I, MHC-II, IL-1R, IL-2Rα, IL-2Rβ, IL-2Rγ, IL-3Rα, CSF2RB, IL-4R, IL-5Rα, CSF 2RB, IL-6Rα, gp130, IL-7Rα, IL-9R, IL-10R, IL-12Rβ1, IL-12Rβ2, IL-13Rα1, IL-13Rα2, IL-15Rα, IL-21R, IL23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LT13R, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAFS, BAFF, BAFF-R, APRIL, TRAIL, RANK, AITR, TRAMP, CCR1, CCR2, CCR3, CCR4, CCRS, CCR6, CCR7, CCR8, CCR9, CCR10, CCR11,CXCR1, CXCR2, CXCR3, CXCR4, CXCRS, CXCR6, CXCR7, CLECL9a, DC-SIGN, IGSF4A, SIGLEC, EGFR, PDGFR, VEGFR, FAP,α-SMA, FAS, FAS-L, Fc, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULTI, RAE1 α, β, γ, δ, and ε, H60a, H60b, H60c, GPR15, and ST2. In embodiments, integrins are selected from one or more of: α1, α1, α2, αllb, α3, α4, α5, α6, α7, α8, α9, α10, α11, αD, αE, αL, αM, αV, αX, β1, β1, β2, β3, β4, β5, β6, β7, β8, or combinations thereof. In embodiments, TCR is selected from one or more of: α, β, γ, δ, ε,ζ, or combinations thereof. Several methods have been described in the literature for assaying of cell-surface protein expression, including Flow Cytometry and Mass Cytometry (CyTOF).

The present disclosure also relates to treating pathologic hyperinflammation associated with CSS and ARDS, comprising administering the negatively charged particles as disclosed herein. In embodiments, negatively charged particles of the present disclosure treat pathological inflammation during ARDS without causing broad immune suppression. In embodiments, negatively charged particles of the present disclosure treat inflammation associated with CSS and ARDS and associated sequalae. Inflammatory monocytes and neutrophils in the periphery can be characterized by the expression of cell-surface markers. Distinct circulating monocyte populations, differentiated by expression of specific cell-surface markers, have been shown to carry out specific effector functions. Inflammatory monocytes exhibit a CD14+CD16− phenotype in humans (CX3CR1Lo CCR2+Gr1+ or CX3CR1Lo CCR2+ Ly6CHi phenotype in mice). Conversely, monocytes having the CD14LoCD16+ phenotype in humans, an CX3CR1HiCCR2-Gr1− or CX3CR1HiCCR2-Ly6CLo monocytes recruited to sites of injury differentiate into mature macrophage populations that promote wound healing and perform anti-inflammatory homeostatic functions. Similarly, inflammatory neutrophils can also be characterized by the expression of cell-surface markers (e.g CD66b and CD63), and production of proteins (e.g myeloperoxidase, neutrophil elastase, gasdermin, cathepsin-G, and peptidyl arginine deaminase 4 (PADI-4)), and protein-DNA complexes called neutrophil extracellular traps (NETs).

In any one of the methods disclosed herein, the negatively charged particles are administered to a subject with abnormal levels of inflammatory monocytes exhibiting a CD14+CD16− phenotype and neutrophils exhibiting a CD15+CD66b+CD63+phenotype. In embodiments, the method disclosed herein reduces or ameliorates the abnormal levels of inflammatory monocytes exhibiting a CD14+CD16− phenotype and neutrophils exhibiting a CD15+CD66b+CD63+ phenotype.

In any one of the methods disclosed herein, the negatively charged particles are administered to a subject with abnormal levels of inflammatory neutrophils. In embodiments, the abnormal levels of inflammatory neutrophil expresses cell surface markers CD66b and/or CD63. In embodiments, the method disclosed herein reduces or ameliorates the abnormal levels of inflammatory neutrophils. In embodiments, the inflammatory neutrophils are characterized by the production of protein such as myeloperoxidase or neutrophil elastase. In embodiments, the inflammatory neutrophils are characterized by protein-DNA complexes called neutrophil extracellular traps (NETs).

The present disclosure also relates to reducing the accumulation of inflammatory monocytes and/or neutrophils in a subject with ARDS or CSS, comprising administering the negatively charged particles as disclosed herein. In several pre-clinical animal models of inflammation and in humans, including in the cases of CSS and ARDS, inflammatory monocytes and neutrophils have been shown to rapidly infiltrate and accumulate at sites of inflammation where their pro-inflammatory activities are associated with life-threatening pathologies.

The present disclosure also relates to the reprogramming of inflammatory monocytes, macrophages, and neutrophils in a patient with ARDS or CSS, comprising administering the negatively charged particles disclosed herein. In embodiments, administering the negatively charged particles reprograms pro-inflammatory monocytes and macrophages into anti-inflammatory monocytes and macrophages. In embodiments, administering the negatively particles reprogram pro-inflammatory monocytes and macrophages from the Ml-type to the anti-inflammatory M2-type. In embodiments, administering the negatively charged particles reduces the production and/or secretion of pro-inflammatory mediators by monocytes and macrophages. In embodiments administering the negatively charged particles reduces the production and/or secretion of pro-inflammatory mediators by monocytes and macrophages by about 10%, 15%, 20%, 25%, 30%, 35%, 40%, 45%, 50%, 55%, 60%, 65%, 70%, 75%, 80%, 85%, 90%, 95%, 99%, or 100% compared to a patient with CSS and/or ARDS that does not receive treatment with the negatively charged particles disclosed herein. In embodiments, administering the negatively charged particles increases the production and/or secretion of anti-inflammatory mediators by monocytes and macrophages. In embodiments, administering the negatively charged particles increases the production and/or secretion of anti-inflammatory mediators by monocytes and macrophages by about 10%, 15%, 20%, 25%, 30%, 35%, 40%, 45%, 50%, 55%, 60%, 65%, 70%, 75%, 80%, 85%, 90%, 95%, 99%, or 100% compared to a patient with CSS and/or ARDS that does not receive treatment with the negatively charged particles disclosed herein. In embodiments, administering the negatively charged particles reprograms pro-inflammatory neutrophils into anti-inflammatory neutrophils. In embodiments, administering the negatively charged particles reprograms pro-inflammatory neutrophils from the N1-type to the anti-inflammatory N2-type. In embodiments, administering the negatively charged particles reduces the production and/or secretion of pro-inflammatory mediators by neutrophils. In embodiments administering the negatively charged particles reduces the production and/or secretion of pro-inflammatory mediators by neutrophils by about 10%, 15%, 20%, 25%, 30%, 35%, 40%, 45%, 50%, 55%, 60%, 65%, 70%, 75%, 80%, 85%, 90%, 95%, 99%, or 100% compared to a patient with CSS and/or ARDS that does not receive treatment with the negatively charged particles disclosed herein. In embodiments, administering the negatively charged particles increases the production and/or secretion of anti-inflammatory mediators by neutrophils by about 10%, 15%, 20%, 25%, 30%, 35%, 40%, 45%, 50%, 55%, 60%, 65%, 70%, 75%, 80%, 85%, 90%, 95%, 99%, or 100% compared to a patient with CSS and/or ARDS that does not receive treatment with the negatively charged particles disclosed herein.

The present disclosure also relates to regulating immune-cell accumulation in the tissues, organs, and/or site(s) inflammation of a CSS and/or ARDS patient, comprising administering the negatively charged particles as disclosed herein. In embodiments, the regulating immune-cell accumulation is reducing immune- cell accumulation. The present disclosure also relates to regulating (e.g., reducing) immune-cell activation in the tissues, organs, and/or site(s) inflammation of a CSS and/or ARDS patient, comprising administering the negatively charged particles as disclosed herein. In embodiments, immune-cell accumulation is reduced by about 10%, 15%, 20%, 25%, 30%, 35%, 40%, 45%, 50%, 55%, 60%, 65%, 70%, 75%, 80%, 85%, 90%, 95%, 99%, or 100% compared to a patient with CSS and/or ARDS that does not receive treatment with the negatively charged particles disclosed herein. In embodiments, the immune-cells are selected from one or more of: monocytes, neutrophils, macrophages, granulocytes, dendritic cells, T-cells, B-cells, NK-cells, and NKT-cells.

The present disclosure also relates to altering immune response, foreign-body response, metabolism, apoptosis, cell death, necrosis, ferroptosis, autophagy, cell migration, endocytosis, phagocytosis, DNA-damage response, pinocytosis, tight-junction regulation, cell adhesion, and/or cellular differentiation in an ARDS patient, comprising administering the negatively charged particles as disclosed herein. The present disclosure also relates to regulating immune response, foreign-body response, metabolism, apoptosis, cell death, necrosis, ferroptosis, autophagy, cell migration, endocytosis, phagocytosis, DNA-damage response, pinocytosis, tight-junction regulation, cell adhesion, and/or cellular differentiation in a CSS and/or ARDS patient, comprising administering the negatively charged particles as disclosed herein.

In embodiments, the present disclosure relates to improving one or more symptoms associated with CSS and/or ARDS, comprising administering the negatively charged particles as disclosed herein. In embodiments, the symptom associated with CSS and/or ARDS includes shortness of breath, rapid breathing (tachypnea), labored breathing, requiring mechanical ventilation, muscle fatigue, general fatigue, low blood pressure, low blood oxygen levels (hypoxemia), discoloration of the skin, discoloration of the nails, respiratory acidosis, hypercapnia, dry cough, fever, chest pain, headache, inflammation of the lungs, fluid buildup in the lungs, atelectasis, crackling or bubbling sound in the lungs, fast pulse rate, dizziness, mental confusion, edema, pulmonary edema, and/or alveolar edema. In embodiments, the symptom associated with CSS and/or ARDS includes one or more selected from: lung inflammation, atelectasis, distressed breathing, fatigue, low blood pressure, fever, headache, hypoxemia, respiratory acidosis, hypercapnia, edema, pulmonary edema, or alveolar edema.

In embodiment, the present disclosure relates to improving one or more symptoms associated with CSS and/or ARDS, the symptom is selected from one or more of: multi-organ dysfunction, brain damage, lung damage, liver damage, kidney damage, heart damage, edema, cerebral edema, pulmonary edema, alveolar edema, respiratory distress, hypoxemia, respiratory acidosis, hypertriglyceridemia, leukopenia, cytopenia, weight loss, or elevated levels of inflammatory markers. In any one of the methods disclosed herein, the present disclosure relates to improving one or more symptoms associated with CSS and/or ARDS, comprising administering the negatively charged particles as disclosed herein. In embodiments, the symptom associated with ARDS includes shortness of breath, rapid breathing (tachypnea), labored breathing, requiring mechanical ventilation, muscle fatigue, general fatigue, low blood pressure, low blood oxygen levels (hypoxemia), discoloration of the skin, discoloration of the nails, respiratory acidosis, hypercapnia, dry cough, fever, chest pain, headache, inflammation of the lungs, fluid buildup in the lungs, atelectasis, crackling or bubbling sound in the lungs, fast pulse rate, dizziness, mental confusion, edema, pulmonary edema, weight loss, and/or alveolar edema. In embodiments, the symptom associated with CSS and/or ARDS includes one or more selected from: lung inflammation, atelectasis, distressed breathing, fatigue, low blood pressure, fever, headache, hypoxemia, respiratory acidosis, hypercapnia, edema, pulmonary edema, weight loss, alveolar edema, or any combination thereof.

In embodiments, the present disclosure relates to improving one or more symptoms associated with CSS and/or ARDS, comprising administering the negatively charged particles as disclosed herein, wherein the symptom is selected from fever, fatigue, swelling of extremities, hepatitis, splenomegaly, loss of appetite, muscle and joint pain, nausea, vomiting, diarrhea, rashes, fast breathing, shortness of breath, ARDS, rapid heartbeat, cough, low blood pressure (hypotension), cytopenia, seizures, headache, lethargy, poor responsiveness, confusion, delirium, hallucinations, tremor, loss of coordination, coagulopathy, multisystem organ dysfunction, multi-organ failure, elevated levels of lactate dehydrogenase (LDH), elevated levels of c-reactive protein, elevated levels of ferritin, elevated levels of pro-inflammatory cytokines, elevated levels of alanine transaminase (ALT), elevated levels of aspartate transaminase (AST), low levels of white blood cells, low levels of lymphocytes, low levels of platelets, low levels of fibrinogen, weight loss, low levels of erythrocyte sedimentation rate (ESR), or any combination thereof.

In embodiments, the present disclosure relates to reducing blood plasma or blood serum AST levels in a CSS and/or ARDS patient, comprising administering the negatively charged particles as disclosed herein. In embodiments, the AST level is reduced to less than about 60 U/I, less than about 59 U/I, less than about 58 U/I, less than about 57 U/I, less than about 56 U/I, less than about 55 U/I, less than about 54 U/I, less than about 53 U/I, less than about 52 U/I, less than about 51 U/I, less than about 50 U/I, less than about 49 U/I, less than about 48 U/I, less than about 47 U/I, less than about 46 U/I, less than about 45 U/I, less than about 44 U/I, less than about 43 U/I, less than about 42 U/I, less than about 41 U/I, less than about 40 U/I, less than about 39 U/I, less than about 38 U/I, less than about 37 U/I, less than about 36 U/I, less than about 35 U/I, less than about 34 U/I, less than about 33 U/I, less than about 32 U/I, less than about 31 U/I, less than about 30 U/I. In embodiments, AST level is reduced to or below the upper limit of normal (ULN). In embodiments, the AST level is reduced to ULN≤40 U/I.

In embodiments, the present disclosure relates to increasing blood plasma or blood serum fibrinogen levels in a CSS and/or ARDS patient, comprising administering the negatively charged particles as disclosed herein. In embodiments, the fibrinogen level is increased to greater than about 100 mg/dl, greater than about 120 mg/dl, greater than about 140 mg/dl, greater than about 160 mg/dl, greater than about 180 mg/dl, greater than about 200 mg/dl, greater than about 220 mg/dl, greater than about 240 mg/dl, greater than about 260 mg/dl, greater than about 280 mg/dl, greater than about 300 mg/dl, greater than about 320 mg/dl, greater than about 340 mg/dl, greater than about 360 mg/dl, or greater than about 340 mg/dl, including all values and ranges therein. In embodiments, the fibrinogen level is increased to the lower limit of normal. In embodiments, the fibrinogen level is increased to the upper limit of normal. In embodiments, the fibrinogen level is increased to between 200 to 400 mg/dL.

In embodiments, the present disclosure relates to improving PaO2/FiO2 ratio in a patient, comprising administering the negatively charged particles as disclosed herein. In embodiments, the method improves PaO2/FiO2 ratio to greater than 100 mmHg. In embodiments, the method improves PaO2/FiO2 ratio to greater than about 200 mmHg. In embodiments, the method improves PaO2/FiO2 ratio to greater than about 250 mmHg. In embodiments, the method improves PaO2/FiO2 ratio to greater than about 300 mmHg. In embodiments, the method improves PaO2/FiO2 ratio to greater than 300 mmHg. The Berlin definition uses the PaO2/FiO2 ratio to distinguish mild ARDS (200 <PaO2/FiO2 <300 mmHg), moderate ARDS (100 <PaO2/FiO2 <200 mmHg), and severe ARDS (PaO2/FiO2 <100 mmHg). See Papazian, L. et al. Ann. Intensive Care (2019) 9:69.

In embodiments, the present disclosure relates to stabilizing or reducing weight loss in a patient having CSS or ARDS. In an embodiment, a patient is administered the negatively charged particles of the disclosure to stabilize or reduce weight loss in a CSS or ARDS patient. In an embodiment, the administration is effective in preventing weight loss as compared to an otherwise comparable method lacking the administration. In an embodiment, the administration is effective in stabilizing weight loss as compared to an otherwise comparable method lacking the administration. In an embodiment, the administration is effective in preventing weight loss by at least about: 1%, 2%, 5%, 10%, 15%, 20%, 25%, 30%, 35%, 40%, 50%, 5-10%, 10-15%, 5-15%, 10-20%, 10-25%, 5-20%, 10-30%, 20-40%, or 15-35% of percent body weight loss as compared to an otherwise comparable method lacking the administration of the negatively charged particles. In an embodiment, the method is effective in preventing weight loss by at least about 1, 2, 3, 4, 5, 6, 7, 8, 9, 10, 11, 12, 13, 14, 15, 20, 25, 30, 35, 40, or 50 days post administration of the negatively charged particles of the disclosure. In an embodiment, the method is effective in preventing weight loss by at least about 5-7 days, 7-14 days, 5-30 days, or at least about 5 days, 7 days, 14 days, 20 days, 30 days, 1 month or 2 months post administration of the negatively charged particles of the disclosure. Weight loss percentage can be determined by taking weight lost (for example pounds) divided by starting weight and multiplying the result by 100.

In embodiments, the present disclosure relates to preventing a CSS and/or ARDS patient from requiring the use of a ventilator, comprising administering the negatively charged particles as disclosed herein. In embodiments, the present disclosure relates to reducing ARDS patient's need for a ventilator support, comprising administering the negatively charged particles as disclosed herein. In embodiments, the present disclosure relates to reducing ARDS patient's need for a mechanical ventilation, comprising administering the negatively charged particles as disclosed herein. In embodiments, the present disclosure relates to shortening the time for a ventilator use in a CSS and/or ARDS patient, comprising administering the negatively charged particles as disclosed herein. In embodiments, the present disclosure relates to reducing CSS and/or ARDS patient's need for life support interventions, comprising administering the negatively charged particles as disclosed herein. In embodiments, the present disclosure relates to improving survival of a CSS and/or ARDS patient, comprising administering the negatively charged particles as disclosed herein. In embodiments, survival may be improved by about 10%, 15%, 20%, 25%, 30%, 35%, 40%, 45%, 50%, 55%, 60%, 65%, 70%, 75%, 80%, 85%, 90%, 95%, 99%, or 100% compared to a patient with CSS and/or ARDS that does not receive treatment with the negatively charged particles disclosed herein.

In embodiments, the present disclosure relates to improving organ function in a CSS and/or ARDS patient, comprising administering the negatively charged particles as disclosed herein. In embodiments, administering the negatively charged particles to a subject with CSS and/or ARDS improves functioning of the lung, liver, kidney, brain, stomach, pancreas, liver, vasculature, eyes, and heart. In embodiments, the present disclosure relates to increasing anti-inflammatory effects in a CSS and/or ARDS patient, comprising administering the negatively charged particles as disclosed herein. In embodiments, the present disclosure relates to increasing anti-inflammatory effects in a patient's lungs, liver, kidney, brain, stomach, pancreas, liver, vasculature, eyes, and heart comprising administering the negatively charged particles as disclosed herein. In embodiments, the present disclosure relates to reducing tissue damage in a CSS and/or ARDS patient, comprising administering the negatively charged particles as disclosed herein. In embodiments, the tissue damage is lung, liver, kidney, brain, stomach, pancreas, liver, vasculature, eye, and heart tissue damage. In embodiments, the present disclosure relates to accelerating immune healing mechanism in a CSS and/or ARDS patient, comprising administering the negatively charged particles as disclosed herein. In embodiments administering the negatively charged particles to a subject with CSS and/or ARDS improves functional recovery. In embodiments, administering the negatively charged particles to a subject with CSS and/or ARDS improves locomotor function, visual function, cardiovascular function, respiratory function, kidney function, and/or cognitive function. Various clinical scoring methods for assessing functional recover have been disclosed in the literature and are incorporated herein by reference (Glasgow Coma Scale; Extended Glasgow Outcomes Scale; WHO Ordinal Outcomes Scale¹¹, Telephone Interview for Cognitive Status (TICS)¹², Berlin Criteria for ARDS¹³, ASIA Spinal Cord Injury Assessment Scale¹⁴, HScore for Reactive Hemophagocytic Syndrome¹⁵, APPS Outcomes Score for ARDS¹⁶, and BIMS Scale for Cognitive Function¹⁷).

### Administering and Dosing

Methods of the disclosure are performed using any medically accepted means for introducing a therapeutic directly or indirectly into a mammalian subject, including but not limited to injections, oral ingestion, intranasal, topical, transdermal, parenteral, inhalation spray, vaginal, or rectal administration. The term parenteral as used herein includes subcutaneous, intravenous, intramuscular, intraarticular, intraperitoneal, intrathecal and intracisternal injections, as well as catheter or infusion techniques. In various embodiments, the particle is administered intravenously, but may be administered through other routes of administration such as, but not limited to: intradermal, subcutaneous, epictuaneous, oral, intra-articular, and intrathecal. In any one of the methods as disclosed herein, the subject is human.

In any one of the method disclosed herein, the negatively charged particles can be administered to a subject in need thereof at a dose in a range of about 0.1 mg/kg to about 10 mg/kg, including all values and ranges that lie in between these values. In embodiments, a dose of the negatively charged particles is in the range of about 1 mg/kg to about 8 mg/kg, about 1.5 mg/kg to about 7 mg/kg, about 1.5 mg/kg to about 6 mg/kg, about 1.5 mg/kg to about 5 mg/kg, about 1.5 mg/kg to about 4.5 mg/kg, about 2 mg/kg to about 4 mg/kg, including all values and ranges that lie in between these values.

In any one of the methods disclosed herein, the negatively charged particles can be administered to a subject in need thereof at a dose in a range of about 1 mg to about 800 mg, including all values and ranges that lie in between these values. In embodiments, a dose of the negatively charged particles is in the range of about 10 mg to about 700 mg, about 10 mg to about 650 mg, about 15 mg to about 650 mg, about 20 mg to about 650 mg, about 25 mg to about 650 mg, about 30 mg to about 650 mg, about 35 mg to about 650 mg, about 40 mg to about 650 mg, about 45 mg to about 650 mg, about 50 mg to about 650 mg, including all values and ranges that lie in between these values. In embodiments, the doses discussed herein are daily doses.

In various embodiments, the particle is administered daily, every other day, twice daily, three times per day, seven times per week, six times per week, five times per week, four times per week, three times per week, twice weekly, once weekly, once every two weeks, once every three weeks, once every 4 weeks, once every two months, once every three months, once every 6 months or once per year, including values and ranges that lie in between.

In various embodiments, the particles are administered as a single dosage form or as multiple dosage forms.

In any one of the methods disclosed herein, the negatively charged particles can be administered to a subject in need thereof once, twice, or three times a day. In embodiments, the negatively charged particle is administered once a day.

In any one of the methods disclosed herein, the negatively charged particles can be administered to a subject in need thereof daily for a duration determined by a physician. In embodiments, the negatively charged particles can be administered to a subject in need thereof two or more time a week, for a duration determined by a physician.

In any one of the methods disclosed herein, the negatively charged particles can be administered prior to onset of CSS and/or ARDS, at the onset of CSS and/or ARDS, or after the onset of CSS and/or ARDS.

In any one of the methods disclosed herein, the negatively charged particles can be administered to a subject in need thereof orally, by inhalation, or intravenously. In embodiments, the negatively charged particle is administered by an IV infusion. In embodiments, the IV infusion is administered over about 30 minutes to about 5 hours, including all values and ranges that lie in between these values. In embodiments, the IV infusion is administered over about 30 minutes to about 4 hours, about 30 minutes to about 3 hours, about 30 minutes to about 2.5 hours, about 1 hour to about 2 hours, including all values and ranges that lie in between these values.

In any one of the methods disclosed herein, the negatively charged particles can be administered to a subject in need thereof, by an IV infusion at a constant infusion rate. In embodiments, the administration by an IV infusion is given where the infusion rate is adjusted during the administration. In embodiments, the IV infusion rate comprises one or more rates selected from: about 10 mL/hr, about 15 mL/hr, about 20 mL/hr, about 25 mL/hr, about 30 mL/hr, about 35 mL/hr, about 40 mL/hr, about 45 mL/hr, about 50 mL/hr, about 55 mL/hr, about 60 mL/hr, about 65 mL/hr, about 70 mL/hr, about 75 mL/hr, about 80 mL/hr, about 85 mL/hr, about 90 mL/hr, about 95 mL/hr, about 100 mL/hr, about 105 mL/hr, about 110 mL/hr, about 115 mL/hr, about 120 mL/hr, about 125 mL/hr, about 130 mL/hr, about 135 mL/hr, about 140 mL/hr, about 145 mL/hr, or about 150 mL/hr, including all values and ranges that lie in between these values.

In any one of the methods disclosed herein, the negatively charged particles can be administered to a subject in need thereof, by an IV infusion where the infusion rate changes more than once. In embodiments, the IV infusion rate changes one, two, or three times during the administration. In embodiments, the IV infusion is given initially at a first infusion rate, changed to a second infusion rate, then changed again to a third infusion rate. In embodiments, the first infusion rate is between about 10 mL/hr to about 40 mL/hr or between about 15 mL/hr to about 25 mL/hr. In embodiments, the first infusion rate is about 20 mL/hr. In embodiments, the second infusion rate is between about 20 mL/hr to about 80 mL/hr or between about 30 mL/hr to about 50 mL/hr. In embodiments, the second infusion rate is about 40 mL/hr. In embodiments, the third infusion rate is between about 40 mL/hr to about 120 mL/hr or between about 70 mL/hr to about 90 mL/hr. In embodiments, the third infusion rate is about 80 mL/hr. In embodiments, the first infusion rate and the second infusion rate are held for a time ranging from about 5 minutes to about 30 minutes or from about 10 minutes to about 25 minutes, including all values and ranges that lie in between these values. In embodiments, the first infusion rate and the second infusion rate are held for about 15 minutes. In embodiments, the third infusion rate is held for a time ranging from about 30 minutes to about 4 hours, from about 30 minutes to about 3 hours, from about 45 minutes to 150 minutes, from about 60 minutes to about 120 minutes, or from about 75 minutes to about 105 minutes, including all values and ranges that lie in between these values. In embodiments, the third infusion rate is held until the infusion is complete. In embodiments, the third infusion rate is held for about 90 minutes.

In any one of the method disclosed herein, the negatively charged particles can be administered to a subject in need thereof, by an IV infusion administered at a first infusion rate of 20 mL/hr for the first 15 minutes, at a second infusion rate of 40 mL/hr for the next 15 minutes, and at a third infusion rate of 80 mL/hr until the infusion is complete (remainder of the time).

### EXAMPLES

### Example 1

### Preparation of Negatively Charged Particles

A solution of commercially available PLGA (50:50) with acid terminated end groups were mixed with water to produce a primary emulsion. The primary emulsion was rapidly mixed with a solution of polyvinyl alcohol and polyacrylic acid to form a secondary emulsion. The solvent of the resulting double emulsion was removed to form a suspension of the negatively charged particles. The negatively charged particles were washed, filtered, and concentrated via tangential flow filtration.

The negatively charged particles were characterized by dynamic light scattering (DLS) analysis and by Malvern Zetasizer Nano ZS (Malvern Instruments, Westborough, Mass.) at a count rate of 2.5×105 counts per second in 18.2 MSΩ water. The average particle size was about 350 to about 750 nm. The zeta potential of the particle was between about −32 and about

### Example 2

### Preparation of Lyophilized Composition Comprising Negatively charged Particles

To the negatively charged particles as prepared by Example 1, D-mannitol, sucrose, sodium citrate dehydrate, and water were added. Lyophilization vials were filled using aseptic technique and partially stoppered then lyophilized. The lyophilized vials were sealed and crimped with aluminum seals. The sealed vials were sterilized by gamma irradiation. The composition has the ingredients in the ratio listed in Table 1.

TABLE 1
Lyophilized Composition

| | Amount |
|---|---|
| Particles according to Example 1 | 80 g |
| D-Mannitol | 80 g |
| Sucrose | 60 g |
| Sodium Citrate Dihydrate | 4.8 g |

### Example 3

### Safety and Toxicology of Negatively charged Particles

Safety and toxicology study in rats were performed with negatively charged particles having average particle size in the range of 350-750 nm with zeta potential of less than -32 mV.

Study in Rats

Toxicological effects were not observed in rats at doses of between 50 mg/kg to 100 mg/kg (Human equivalent dose level of between 8 mg/kg and 16 mg/kg). All effects were non-adverse and reversible.

The negatively charged particles showed no evidence of complement activation, hemolysis, coagulation, T-cell activation, or liver toxicity in the National Cancer Institute-Nanotechnology Characterization Laboratory (NCI-NCL) in vitro assays.

### Example 4

### Efficacy of Negatively charged Particles at Preventing Pathology Associated with LCMV Infection

The efficacy of negatively charged ONP-302 at resolving viral infection induced systemic inflammation and associated pathologies was examined in a mouse model of primary lymphocytic choriomeningitis virus (LCMV) infection. In the primary LCMV infection model, intravenous injection of LCMV results in systemic infection causing systemic inflammation and weight loss. Additionally, mice fail to clear the infection due to T cell exhaustion and inefficient anti-viral effector immune function.

ONP-302 particles used in this study had an average diameter of 350-750 nm and a zeta potential between −32 and −50 mV.

Briefly, C57BL/6 mice were infected with 2×10⁶ plaque forming units (pfu) LCMV (clone 13) via intravenous tail vein injection. On Day 5 post-infection, mice were randomized into one of the three following groups:

a. Saline on 5 consecutive days.

b. ONP-302 (1 mg/mouse) on 5 consecutive days.

c. ONP-302 (1 mg/mouse) once every 3 days (5 total doses).

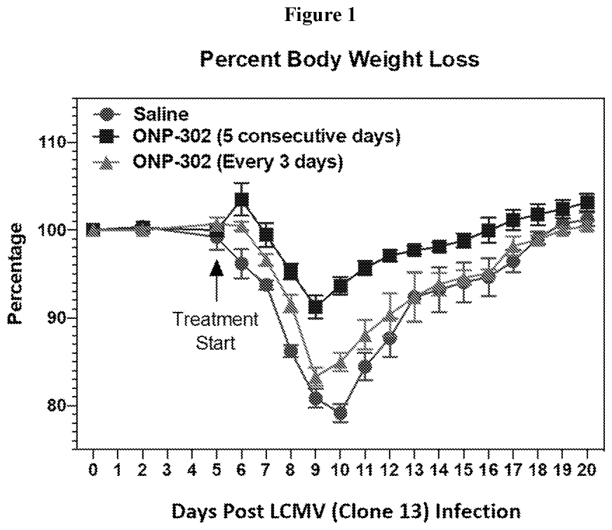

All treatments were administered intravenously via tail vein injection. As shown in FIG. 1, treatment with ONP-302 on 5 consecutive days demonstrated optimal efficacy leading to significantly reduced weight loss compared to the Saline and ONP-302 treatment (n=5 per treatment group) administered once every 3 days.

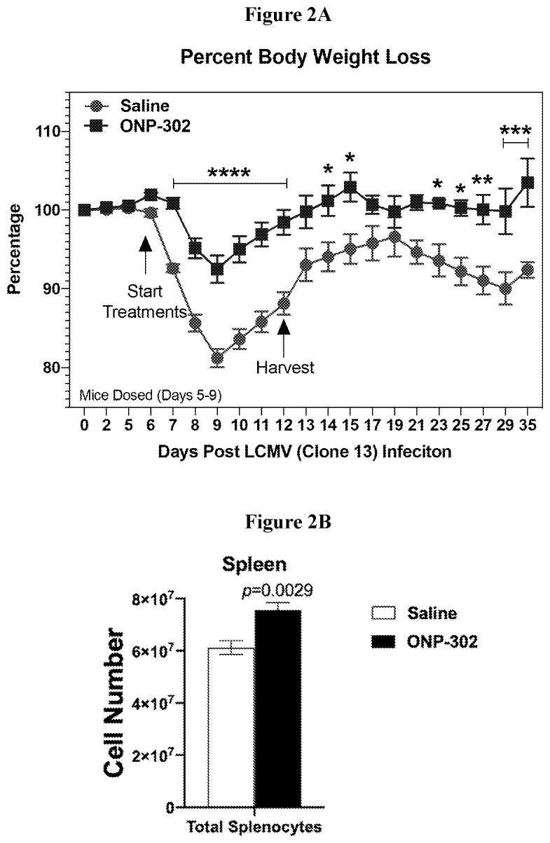

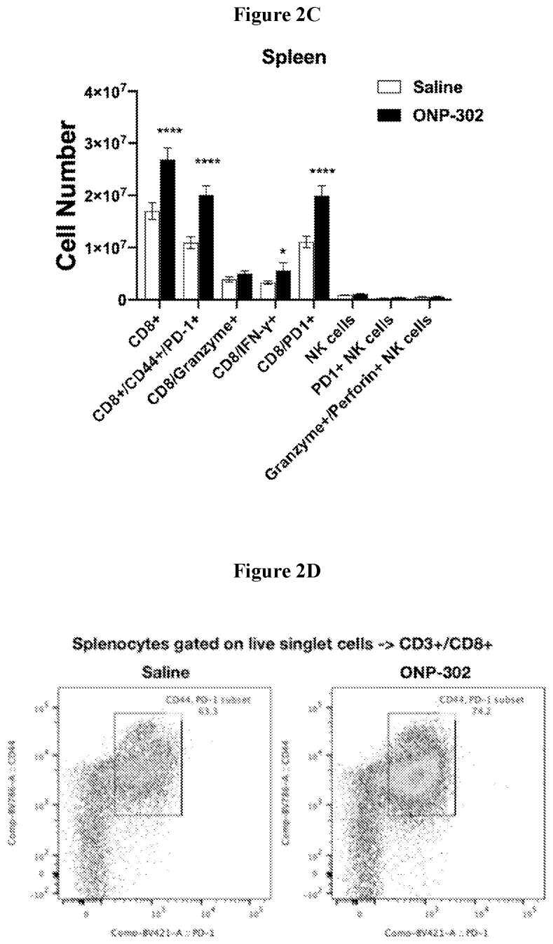

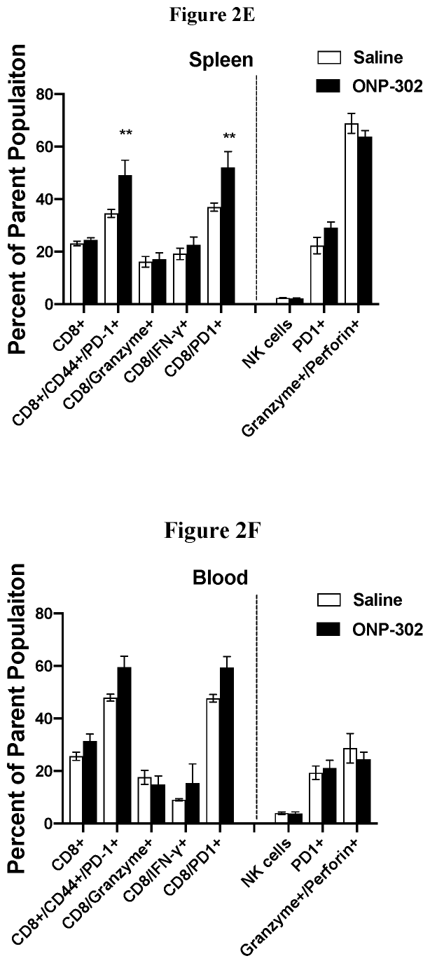

Next, the effect of ONP-302 on the immune system and its ability to clear the systemic viral infection was examined. C57BL/6 mice were infected with 2×10⁶ pfu LCMV (clone 13) via intravenous tail vein injection. On Day 5 post-infection, mice were treated with Saline or ONP-302 (1 mg/mouse) via intravenous tail vein injections on 5 consecutive days (Days 5-9). Mice were followed for weight loss until Day 35 and as seen previously, treatment with ONP-302 led to significantly reduced weight loss compared to Saline treatment (FIG. 2A).

On Day 12 post-infection in this second disease course, a subset of mice was sacrificed from each treatment group and splenocytes were assayed by flow cytometry. As shown in FIG. 2B, treatment with ONP-302 led to a significant increase in the total number of splenocytes. As shown in FIG. 2C, compared to Saline treatment, treatment with ONP-302 led to a significant increase in the total number of CD8 T cells, CD8⁺/CD44⁺/PD1⁺ T cells, CD8⁺/IFN-γ⁺ T cells, and CD8⁺/PD1⁺ T cells. Of note, the CD44hi/PD-1+ T cell population has previously been reported to be LCMV-specific in this model.

Flow cytometry plots depicting the percentage of activated CD8⁺/CD44⁺/PD1⁺ T cells in spleen are shown in FIG. 2D. As shown in FIG. 2E and FIG. 2F, ONP-302 treatment did not alter the percentage of CDS⁺ T cells or activated CDS⁺ T cells in the spleen and blood.

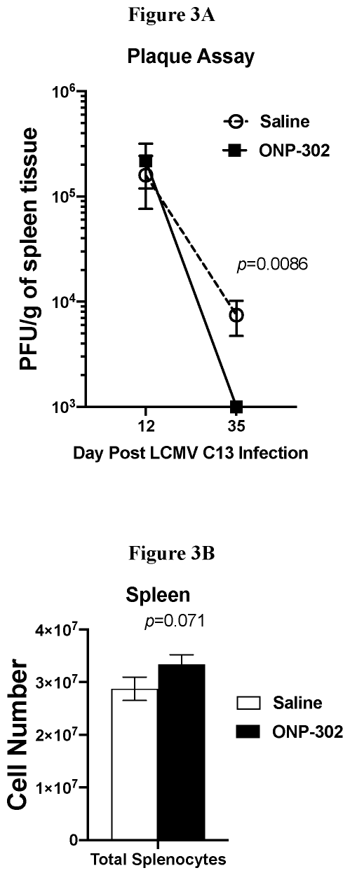

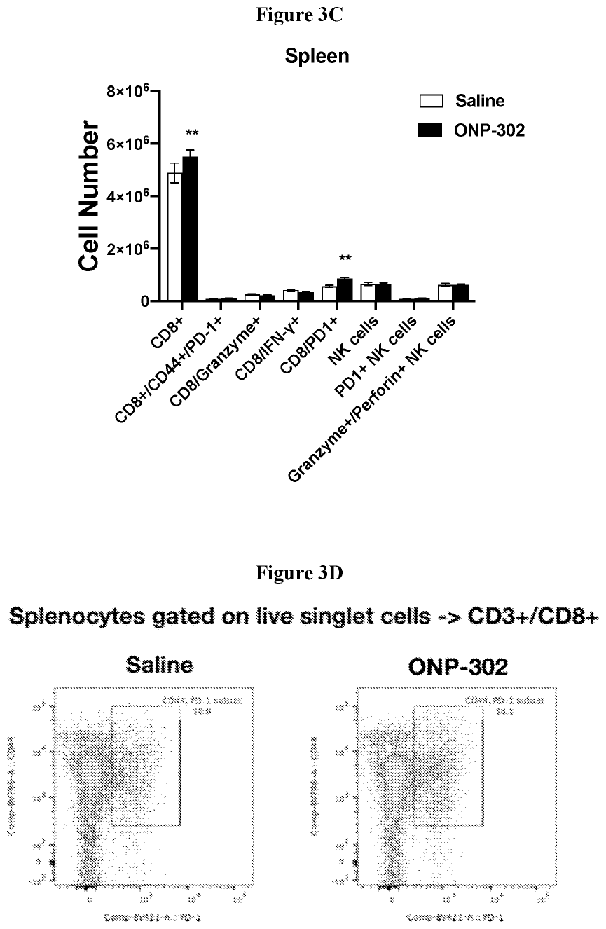

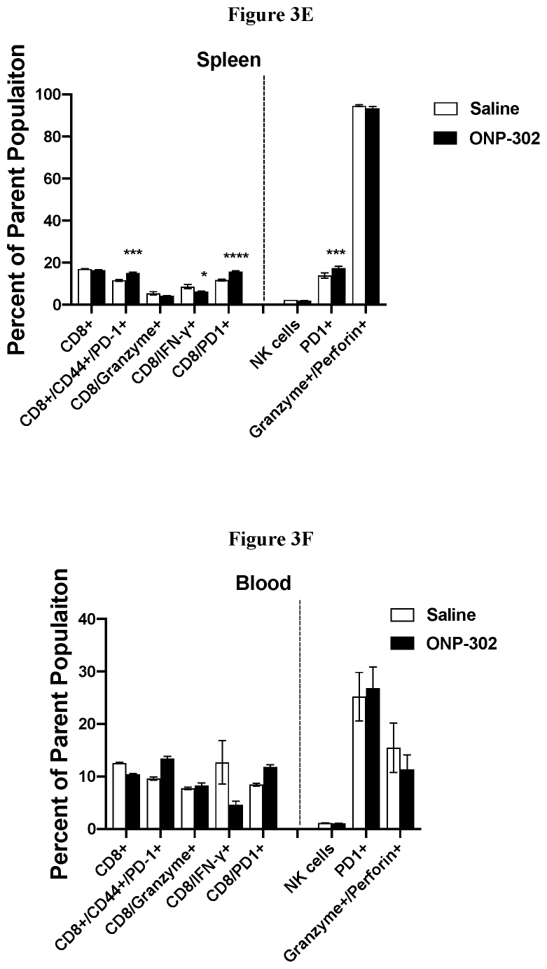

LCMV viral titers were determined in spleen by plaque assay on Days 12 and 35 post-infection. As shown in FIG. 3A, treatment with ONP-302 did not alter the viral titer on Day 12 but led to a statistically significant reduction in viral titers on Day 35 when compared to Saline treatment.

On Day 35, Splenocytes were assayed by flow cytometry to examine the effect of ONP-302 on immune cells at this timepoint. As shown in FIG. 3B, treatment with ONP-302 led to a trend in increased number of cells in the spleen but did not reach statistical significance (p=0.071). As shown in FIG. 3C, treatment with ONP-302 led to a significant increase in the total number of CDS⁺T cells and CD⁺/PD-1⁺ T cells in the spleen. As shown in FIG. 3D, treatment with ONP-302 led to a significant increase in the percentage of CD8⁺/CD44⁺/PD1⁺ T cells, CD8⁺/IFN-γ⁺ T cells, and total PD-1⁺ cell population in spleen compared to Saline. No differences were observed in blood (FIG. 3E).

In conclusion, the results of these experiments demonstrate that:

a. ONP-302 is effective at preventing weight loss after primary LCMV infection.

b. Optimal ONP-302 efficacy is observed when treatment is administered on 5 consecutive days beginning Day 5 post-infection.

c. ONP-302 treatment leads to anti-viral immune activation characterized by increase in activated CDS⁺ T cells in the spleen. This activated CDS⁺ T cell response is associated with improved viral clearance and reduced LCMV viral titers which otherwise persists chronically.

### Example 5

### Efficacy of Negatively charged Particles at Preventing Pathology Associated with H1N1 Infection

The efficacy of negatively charged ONP-302 at resolving viral infection induced acute inflammation and associated lung immunopathology was examined in a mouse model of H1N1 influenza infection using aged mice. In this model in aged mice, H1N1 infection-induced primary lung damage induces an immune response leading to the rapid influx of pro-inflammatory myeloid-derived cells (e.g monocytes and neutrophils) into the lung. At the lung, these cells produce massive quantities of pro-inflammatory mediators which causes excessive inflammation leading to secondary lung damage and associated with poor lung function.

ONP-302 particles used in this study had an average diameter of 350-750 nm and a zeta potential between -32 and -50 mV.

Briefly, 18-22-month-old female C57BL/6 mice were anaesthetized and intranasally infected with 600 pfu H1N1 influenza virus. Beginning on Day 3 post-infection (p.i), mice were randomized into one of the two groups as follows:

a. Saline

b. ONP-302 (1 mg/mouse)

All treatments were administered via tail vein injection. Treatments were administered once daily on 5 consecutive days (Days 3-7). To assess the effect of ONP-302 treatment on lung function, pulse oximetry assessments were performed daily and oxygen saturation in blood was recorded for both treatment groups. Oxygen saturation level in blood determined by pulse oximetry is a clinically relevant measure of lung function and is used routinely for monitoring the lung function of human subjects.

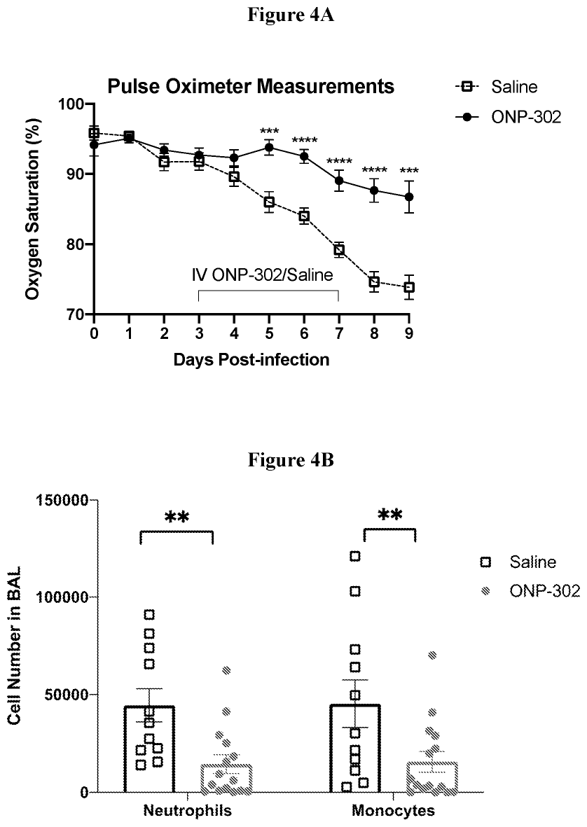

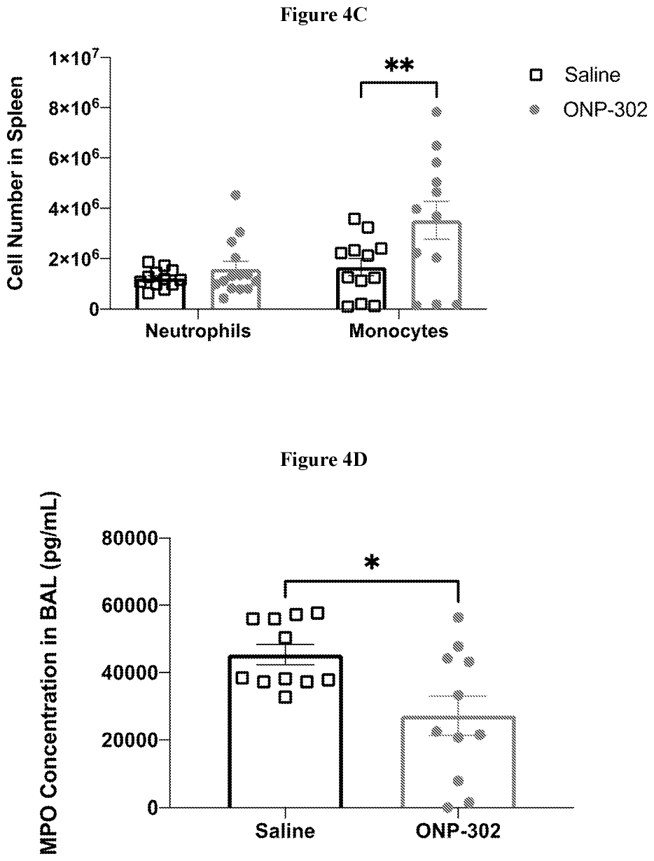

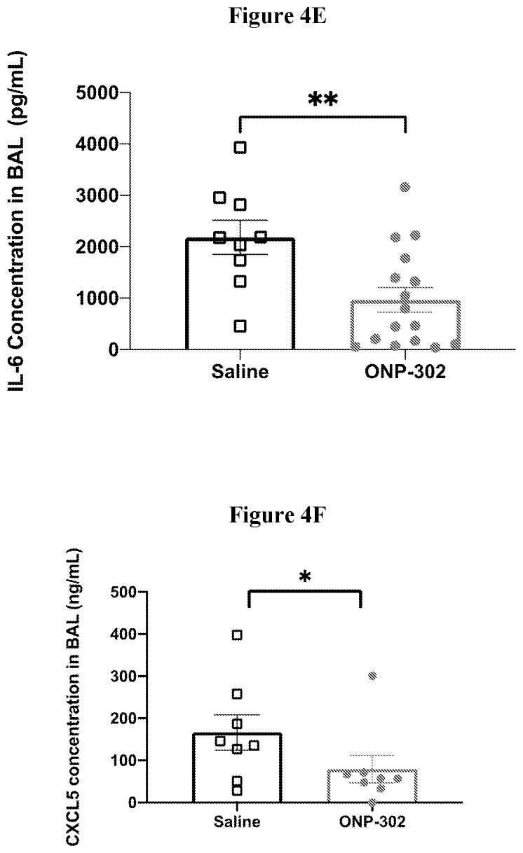

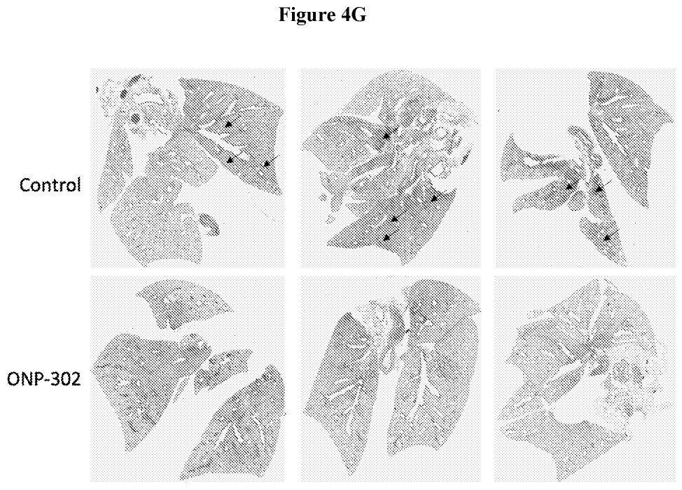

As shown in FIG. 4A, compared to Saline (Control), treatment with ONP-302 lead to a statistically significant improvement in lung function on Days 5-9 p.i as determined by oxygen saturation assessments via pulse oximetry.

Next, the efficacy of ONP-302 at preventing lung inflammation was examined. On Day 9 p.i, mice were sacrificed, and the levels of pro-inflammatory myeloid-derived cell infiltrate, pro-inflammatory cytokine/chemokines, and markers of cellular damage were determined from the assay of bronchoalveolar lavage (BAL) which is a method for assaying the lower respiratory system.

As shown in FIG. 4B, treatment with ONP-302 lead to a statistically significant reduction in the level of total (CD45⁺/CD1 lb⁺) and inflammatory monocytes (CD45⁺/CD11b⁺/Ly6C⁺) and trend towards reduced neutrophils (p=0.06) in the bronchoalveolar lavage (BAL) when compared to Saline. Instead, these cells appeared to be sequestered in the spleen (FIG. 4C) and consistent with the known mechanism of action of ONP-302. As shown in FIG. 4D and FIG. 4E, treatment with ONP-302 lead to a statistically significant reduction in the levels of pro-inflammatory proteins MPO and CXCL-5 in the BAL. As shown in FIG. 4D, 4E, and 4F treatment with ONP-302 lead to a statistically significant reduction in the levels of pro-inflammatory proteins MPO, IL-6, and CXCL-5 in the BAL. As shown in FIG. 4G, treatment with ONP-302 led to reduced lung damage indicated by a statistically significant reduction in the levels of lung damage marker Albumin.

To determine whether inhibition of lung inflammation also prevented lung damage, lung tissues were harvested from mice treated with Saline or ONP-302 on Day 9 p.i. and histopathological assessments were performed. As shown in FIG. 411, significant lung tissue damage was observed in the Saline (Control) treated mice; however, lung tissue appeared to be preserved with reduced damage observed in the ONP-302 treated mice.

In conclusion, results from this study demonstrate that ONP-302 treatment is effective at reducing lung immunopathology and improving lung function after H1N1 infection in aged mice.

### EXAMPLE 6

### Efficacy of Negatively charged Particles at Inhibiting Pro-inflammatory Cytokine Production from PBMCs stimulated with LPS ex vivo.

Lipopolysaccharide (LPS) is a bacterial cell wall component and an endotoxin which gives rise to sepsis-induced cytokine storm. The efficacy of negatively charged particles ONP-302 at inhibiting pro-inflammatory cytokine production from human peripheral blood mononuclear cells (PBMCs) isolated from healthy human subjects was examined ex vivo after incubation with LPS.

Freshly isolated PBMCs were incubated with different concentrations of CNP-301 for 30 minutes and then stimulated with 0.1 ng/mL LPS for 6, 12, or 24 hours in an ex vivo culture. After incubation, cell culture supernatants were collected and the levels of pro-inflammatory cytokines and chemokines (IL-1(3, TNF-α, and MCP-1) were assayed by ELISA. Unstimulated PBMCs were used as controls.

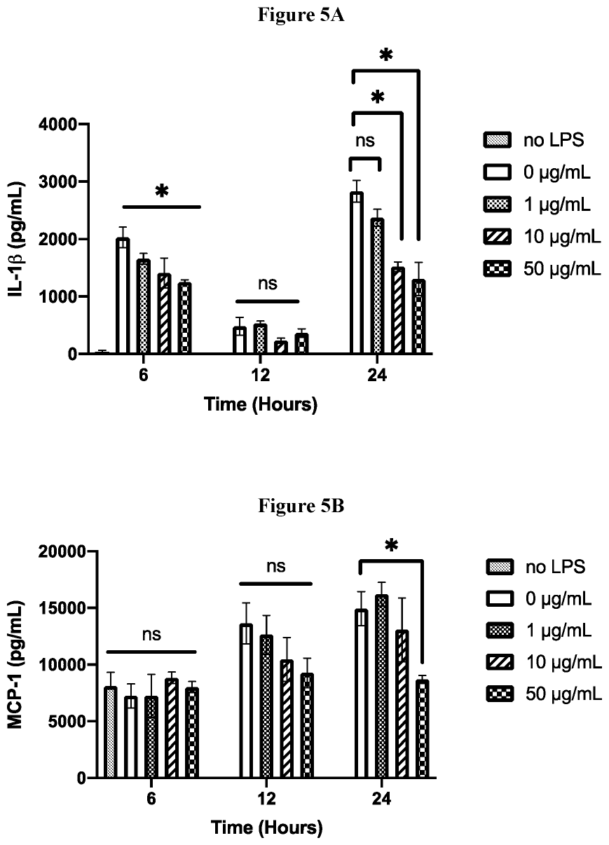

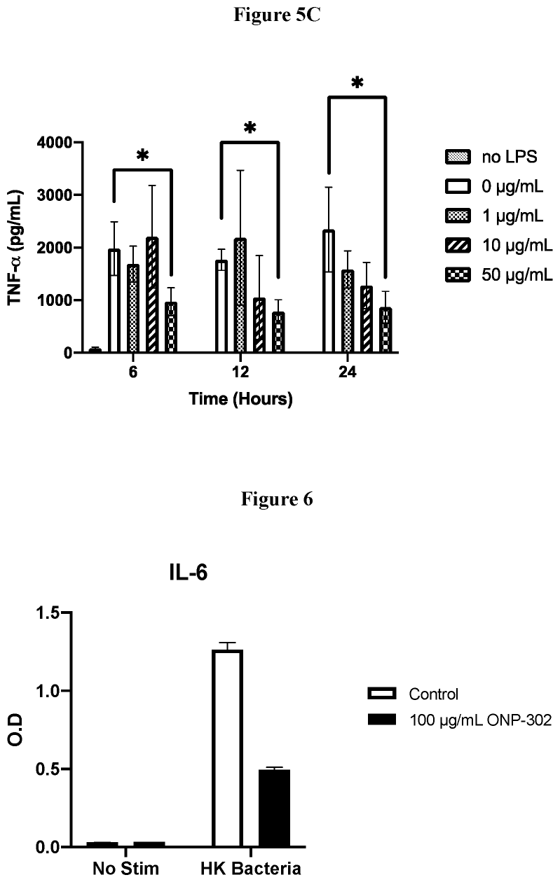

As shown in FIG. 5A, incubation of 50 μg/mL CNP-301 with LPS-stimulated PBMCs for 6 and 24 hours led to a statistically significant reduction in the levels of IL-lB concentrations by 46% and 54%, respectively

As shown in FIG. 5B, incubation of 50 μg/mL CNP-301 with LPS-stimulated PBMCs for 24 hours led to a statistically significant 42% reduction in the levels of MCP-1 in the cell culture supernatants.

As shown in FIG. 5C, incubation of 50 μg/mL CNP-301 with LPS-stimulated PBMCs for 6, 12, or 24 hours led to statistically significant reduction in the levels of TNF-α by 50%, 55%, and 62%, respectively.

In conclusion, these data demonstrate the efficacy of negatively charged particles at inhibiting pro-inflammatory cytokine/chemokine production from PBMCs after incubation with LPS ex vivo suggesting the potential to treat CSS and/or ARDS resulting from sepsis and bacterial infections in vivo.

### Example 7

### Efficacy of Negatively charged Particles at Inhibiting Pro-inflammatory Cytokine Production from Monocytes stimulated with Heat-Killed Bacteria in vitro.

Bacterial infections are associated with induction of a pro-inflammatory response from innate immune cells (e.g monocytes) via TLR2 signaling which can progress to CSS and/or ARDS. To determine whether negatively charged particles could prevent pro-inflammatory cytokine production from monocytes after exposure to bacteria, the efficacy of CNP-301 particles at inhibiting the production of pro-inflammatory cytokine IL-6 from monocytes stimulated with heat-killed bacteria was assessed in vitro. The human monocyte cell-line Mono-Mac-06 and PyroMAT® assay kit (Millipore Sigma) were used in this assay.

Briefly, Mono-Mac-06 cells were co-incubated with 100 μg/mL CNP-301 and heat-killed Staphylococcus aureus (HKSA) for 24 hours according to the manufacturer's instructions. Unstimulated cells and Saline were used as a negative control. 24-hours after incubation, the cell culture supernatant was harvested, and levels of IL-6 were assayed by ELISA according to the manufacturer's instructions.

As shown in FIG. 6, of cells with HKSA in the absence of CNP-301 resulted in robust induction of IL-6 production when compared to unstimulated cells. In contrast, CNP-301 treatment resulted in >50% inhibition of IL-6 production from cells incubated with HKSA.

In conclusion, results from this study suggest that negatively charged particles inhibit pro-inflammatory IL-6 production from monocytes after stimulation with bacteria in vitro and may be useful in preventing CSS and/or ARDS in vivo after bacterial infections.

### Example 8

### Two-part Phase 1b/2a Study to Evaluate Safety and Tolerability of Negatively charged ONP-302 particles.

The present disclosure describes a two-part Phase 1b/2a study to evaluate safety and tolerability of negatively charged ONP-302 particles in an open-label Sentinel Cohort in Part A followed by a randomized placebo-controlled Part B to evaluate safety, tolerability, and efficacy in hospitalized adults with systemic inflammation, sepsis, and/or pneumonia associated with respiratory viral infections (e.g Influenza and SARS-CoV-2).

Part A includes an open-label, repeat dose study of ONP-302 in a Sentinel Cohort of a minimum of 3 subjects. Part B will follow as a randomized, double-blind, repeat dose study using the maximum tolerated dose (MTD) of ONP-302 determined in Part A.

Subjects eligible for inclusion in the study will have the following characteristics:

a. Confirmed positive test result for a respiratory viral infection (e.g., Influenza and SARS-CoV2).

b. Hospitalized with known respiratory viral infections with or without low flow oxygen therapy <6 L/minute (WHO COVID score 3 or 4).

c. Signs of inflammation at Screening characterized by serum c-reactive protein levels ≥40 mg/L, and either serum ferritin levels of ≥300 ng/mL or serum D-dimer ≥0.75 μg/mL.

In Part A, eligible subjects will be enrolled into the Sentinel Cohort immediately prior to the first dose administration (Day 1) of ONP-302. A minimum of three subjects will be enrolled in the Sentinel Cohort. Enrollment of each subject in the sentinel cohort will not proceed until the Safety Committee has reviewed all available safety data through 24 hours post dosing from the previous subject and provided recommendations for continued dosing. Subjects in the Sentinel Cohort will receive five administrations of ONP-302. Subjects will receive a maximum ONP-302 dose of 400 mg (up to 5 mg/kg maximum) based on weight on Day 1. ONP-302 dose level for a subject will be selected based on subject weight on Day 1 according to the following table:

ONP-302 Dose Selection Based on Weight on Day 1

| | | ONP-302 | ONP-302 | Concentration |
|---|---|---|---|---|
| | | Flat | Maximum | in 200 mL |
| Mass | Weight | Dose | Dose | Saline (mg/mL) |
| ≥80 kg | ≥175 lbs. | 400 mg | 5 mg/kg | 2.0 |
| 60-79.9 kg | 130-174.9 lbs. | 300 mg | 5 mg/kg | 1.5 |
| 40-59.9 kg | 85-129.9 lbs. | 200 mg | 5 mg/kg | 1.0 |

Subjects will receive ONP-302 on 5 consecutive days (Days 1-5) via intravenous infusion. Study drug will be administered by intravenous infusion over approximately 3-4 hours, unless safety concerns require premature discontinuation in a subject. The maximum concentration of ONP-302 infused shall not exceed 2.0 mg/mL. Subjects will be observed for acute Adverse Events (AEs), including infusion reactions (IRs), for up to 2 hours following infusion on Days 1-5.

In Part B, eligible subjects will be randomized 1:1 ratio to receive either ONP-302 at the MTD level of ONP-302 determined from Part A or to placebo in a parallel arm design. Approximately 40 subjects will be enrolled in Part B. Subjects will receive five administrations of ONP-302 or placebo (normal saline), for 5 consecutive days (Days 1-5). Study drug will be administered by intravenous infusion over approximately 3-4 hours, unless safety concerns require premature discontinuation in a subject. The maximum concentration of ONP-302 infused shall not exceed 2.0 mg/mL. Subjects will be observed for acute AEs, including IRs, for up to 2 hours following infusion on Days 1-5.

ONP-302 will be administered once daily for 5 consecutive days by intravenous (IV) infusion lasting approximately 3-4 hours using a graduated rate of infusion as follows:

a. 20 mL/hr for the first 15 min,

b. 40 mL/hr for the next 15 min,

c. 80 mL/hr for the remainder of the infusion.

The following will serve as study endpoints:

Safety Endpoints:

a. Frequency of Adverse Events (AEs) and Serious Adverse Events (SAEs).

b. Laboratory Safety Assessments (hematology, serum chemistry, coagulation panel, urinalysis).

c. Physical Examinations including vital signs (blood pressure, heart rate, temperature) and 02 saturation.

d. 12-lead electrocardiogram (ECG).

e. Complement and cytokines/chemokines (collected samples will be analyzed in the event of infusion reaction or other related infusion reaction adverse events with putative complement and cytokine/chemokine involvement.)

Pharmacodynamic Endpoints (Day 1 pre-dose assessments to serve as baseline):

a. Serum C-reactive protein (CRP).

b. Absolute counts of lymphocytes in blood.

c. Clinical Efficacy Endpoints (Day 1 pre-dose assessments to serve as baseline):

d. Composite of COVID Ordinal Outcomes Scale as defined as:

e. Days in hospital

f. Mortality rate

Exploratory Endpoints (Day 1 pre-dose assessments to serve as baseline):

a. Serum d-dimer

b. Serum ferritin.

c. Inflammatory cytokines and chemokines (IL-1β, IL-2, IL-6, IL-7, IL-8, IL-10, TNF-α, IFN-γ, IP-10, MIP-1β, MCP-1, and GM-CSF)

d. Neutrophil to lymphocyte ratio

e. Ventilator-free days

f. SpO₂/FiO₂

g. Lung function as determined by CT scan

Exploratory PK Endpoint: To determine the plasma concentration of ONP-302 PD cytokine biomarker IL-8 pre-dose and at 0.5, 1, 2, 3, 4, and 8 hours after intravenous administration of ONP-302 on Day 1.

### Numbered Embodiments

Notwithstanding the appended claims, the following numbered embodiments also form part of the instant disclosure.

1. A method treating acute respiratory distress syndrome (ARDS) in a subject comprising administering to the subject a therapeutically effective amount of surface-functionalized particles having a negative zeta potential, wherein the surface-functionalized particles are free from other therapeutically active agents.

2. The method of embodiment 1, wherein ARDS is the result of direct lung-injury or indirect lung-injury.

3. The method of embodiment 1, wherein ARDS is the result of pneumonia, lung inflammation, bacterial infection, viral infection, fungal infection, opportunistic infection, sepsis, aspiration of gastric contents, traumatic injury, burn injury, pancreatitis, pulmonary contusion, hemorrhagic shock, near drowning, blood transfusion, inhalation injury, or combinations thereof.

4. The method of embodiment 3, wherein the viral infection is due to a DNA virus, an RNA virus, or a retrovirus.

5. The method of embodiment 4, wherein the DNA virus is a single-stranded DNA (ssDNA) virus, or a double-stranded (dsDNA) virus, and the RNA virus is a double-stranded RNA virus, a single-stranded RNA (ssRNA) (+) virus, ssRNA (−) virus, or a circular ssRNA virus.

6. The method of embodiments 3 or 4, wherein the virus is a respiratory virus.

7. The method of embodiment 3 or 4, wherein the virus is selected from the group consisting Adeno-associated virus, Aichi virus, Australian Bat Lyssavirus, BK polyomavirus, Banna virus, Barmah forest virus, Bunyavirus snowshoe hare, Cercopithecine herpes virus, Chandipura virus, Chikungunya virus, Cosavirus A, Cowpox virus, Coxsackievirus, Crimean-Congo hemorrhagic fever virus, Coronavirus, Dengue virus, Dhori virus, Dugbe virus, Duvenhage virus, Eastern equine encephalitis virus, Echovirus, Encephalomyocarditis virus, Epstein-Barr virus, European Bat Lyssavirus, GB virus C/Hepatitis G virus, Hantaan virus, Hendra virus, Hepatitis A virus, Hepatitis B virus, Hepatitis C virus, Hepatitis delta virus, Human adenovirus, Human astrovirus, Human coronavirus, Human cytomegalovirus, Human enterovirus 68,70, Human herpesvirus 1, Human herpesvirus 2, Human herpesvirus 6, Human herpesvirus 7, Human Immunodeficiency Virus (HIV), Human papillomavirus 1, Human papillomavirus 2, Human papillomavirus 16,18, Human parainfluenza virus, Human parvovirus B19, Human respiratory syncytial virus, Human rhinovirus, Human SARS coronavirus, Human spumarterovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro visu, MERS coronavirus, Measles virus, Mengo encephalomyocarditis, Merkell cell polyomavirus, Mokola virus, Molluscum contagiosum virus, Monkeypox virus, Mumps virus, Murray valley encephalitis virus, New York virus, Nipah virus, Norwalk virus, O'Nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama, Salivirus A, Sandfly fever sicilian virus, Sapporo virus, SARS coronavirus-2, Semliki forest virus, Seoul virus, Simian foamy virus, Simian virus 5, Sindbis virus, Southampton virus, St. Louis encephalitis virus, Tick-borne Powassan virus, Torque teno virus, Toscana virus, Uukuniemi virus, Varicella-zoster virus, Variola virus, Venezuelan equine encephalitis virus, WU polyomavirus, Yaba Monkey tumor virus, Yaba-like disease virus, Yellow fever virus, or Zika virus.

8. The method of embodiment 3, wherein the bacterial infection is due to Staphylococcus, Streptococcus, Mycobacterium, Bacillus, Salmonella, Vibrio, Spirochete, Neisseria, Diplococcus, Pseudomonas, Clostridium, Treponaema, Spirillum, or combinations thereof.

9. The method of embodiment 1, wherein the surface-functionalized particles comprise one or more of: polyglycolic acid (PGA), polylactic acid (PLA), polystyrene, poly (lactic-co-glycolic acid) (PLGA), chitosan, polysaccharide, a lipid, diamond, iron, zinc, cadmium, gold, or silver.

10. The method of embodiment 8, wherein the surface-functionalized particles are poly (lactic-co-glycolic acid) (PLGA) particles.

11. The method of embodiment 9, wherein the PLGA particles comprise a ratio of poly lactic acid:poly glycolic acid ranging from about 90:10 to about 10:90, from about 50:50 to about 90:10, about 50:50 to about 80:20; from about 90:10 to about 50:50, or from about 80:20 to about 50:50.

12. The method of embodiment 9, wherein the surface-functionalized particles comprise 50:50 poly lactic acid:poly glycolic acid.

13. The method of embodiment 1, wherein the surface-functionalized particles comprise carboxyl groups on the particle's surface.

14. The method of embodiment 1, wherein the surface-functionalized particles have a zeta potential ranging from about −100 mV and about −1 mV.

15. The method of embodiment 13, wherein the surface-functionalized particles have a zeta potential ranging from about −80 mV and about −30 mV.

16. The method of embodiment 1, wherein the surface-functionalized particles have a mean diameter ranging from about 0.1 μm to about 10 μm.

17. The method of embodiment 16, wherein the diameter of the surface-functionalized particle ranges from about 400 nm to about 800 nm.

18. The method of embodiment 1, wherein administering the surface-functionalized particles in a subject improves one or more symptoms associated with ARDS.

19. The method of embodiment 18, wherein the one or more symptoms associated with ARDS are selected from: lung inflammation, atelectasis, distressed breathing, fatigue, low blood pressure, fever, headache, hypoxemia, respiratory acidosis, hypercapnia, edema, pulmonary edema, and alveolar edema.

20. The method of embodiment 18, wherein administering the surface-functionalized particles reduces the accumulation of inflammatory mediators in the lungs.

21. The method of any of the preceding embodiments, wherein the surface-functionalized particles are administered intravenously.

22. A method of treating acute inflammation in a subject comprising administering negatively charged particles that are free from attached or encapsulated drug, wherein the particles are administered at a dose of between 0.1 mg/kg to 10 mg/kg.

23. A method of treating acute inflammation in a subject comprising administering negatively charged particles free from attached or encapsulated drug, wherein the particles are administered at a dose of between 10 mg to 1000 mg.

24. The method of any one of embodiments 22-23, wherein the negatively charged particles are administered intravenously, subcutaneously, intramuscularly, intraperitoneally, intranasally, or orally.

25. The method of any one of embodiments 22-24, wherein the negatively charged particles are administered as a single dose.

26. The method of any one of embodiments 22-24, wherein the negatively charged particles are administered in multiple doses.

27. The method of any one of embodiments 22-26, wherein the negatively charged particles comprise Poly (lactic-co-glycolic acid) (PLGA), poly (lactic acid) (PLA), poly (glycolic acid) (PGA), polystyrene, chitosan, polysaccharide, one or more lipids, diamond, iron, zinc, cadmium, gold, or silver.

28. The method of any one of embodiments 22-27, wherein the negatively charged particles comprise one or more biodegradable polymers.

29. The method of any one of embodiments 22-28, wherein the negatively charged particles comprise PLGA.

30. The method of any one of embodiments 22-29, wherein the negatively charged particles have a negative zeta potential.

31. The method of any one of embodiments 22-30, wherein the negatively charged particles have a zeta potential is between about 0 mV and -100 mV.

32. The method of any one of embodiments 22-31, wherein the negatively charged particles have a zeta potential is between about −30 mV and -80 mV.

33. The method of any one of the preceding embodiments, wherein the surface functionalized particles have an average diameter between 0.3μm and 3μm.

34. The method of embodiment 24, wherein the negatively charged particles are administered via intravenous infusion over a duration of 0.5, 1, 2, 3, 4, 5, 6, 7, or 8 hours.

35. The method of embodiment 24, wherein the negatively charged particles are administered via intravenous infusion over a duration of <4 hours.

36. The method of embodiment 24, wherein the negatively charged particles are administered via intravenous infusion over a duration of 3-4 hours.

37. The method of embodiment 26, wherein the multiple doses of negatively charged particles are administered once daily on consecutive days.

38. The method of embodiment 37, wherein the multiple doses of negatively charged particles are administered once daily on 2, 3, 4, 5, 6, 7, 8, 9, or 10 consecutive days.

39. The method of any one of the preceding embodiments, wherein the IMP is administered at a concentration of <12.5 mg/mL.

40. The method of embodiment 39, wherein the negatively charged particles are administered at a concentration of 0.05 mg/mL, 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL.

41. The method of any one of the preceding embodiments, wherein the negatively charged particles are administered beginning 0.5, 1, 2, 3, 4, 5, 6, 7, 8, 12, 24, 48, 72, or 96 hours after the onset of acute inflammation.

42. The method of embodiment 22, wherein the subject has acute inflammation resulting from one or more infections, traumatic brain injury, concussion, spinal cord injury, burn injury, ischemic injury, reperfusion injury, sepsis, cytokine release syndrome, pancreatitis, pulmonary contusion, acute respiratory distress syndrome, hemorrhagic shock, inhalation, pneumonia, injury, macrophage activation syndrome, reactive hemophagocytic syndrome, secondary hemophagocytic syndrome (sHLH), severe inflammatory response syndrome (SIRS), cell therapies, severe acute respiratory syndrome (SARS), or combinations thereof.

43. The method of embodiment 42, wherein the infections comprise one or more viral, bacterial, fungal, prion, or opportunistic infections.

44. The method of embodiment 43, wherein the virus is a DNA virus, an RNA virus, or a retrovirus.

45. The method of embodiment 44, wherein the DNA virus is a single-stranded DNA (ssDNA) virus, or a double-stranded (dsDNA) virus, and wherein the RNA virus is a double-stranded RNA virus, a single-stranded RNA (ssRNA) (+) virus, ssRNA (−) virus, or a circular ssRNA virus.

46. The method of embodiments 43-45, wherein the virus is a respiratory virus.

47. The method of any one of embodiments 43-46, wherein the virus is selected from: Adeno-associated virus, Aichi virus, Australian Bat Lyssavirus, BK polyomavirus, Banna virus, Barmah forest virus, Bunyavirus snowshoe hare, Cercopithecine herpes virus, Chandipura virus, Chikungunya virus, Cosavirus A, Cowpox virus, Coxsackievirus, Crimean-Congo hemorrhagic fever virus, Coronavirus, Dengue virus, Dhori virus, Dugbe virus, Duvenhage virus, Eastern equine encephalitis virus, Echovirus, Encephalomyocarditis virus, Epstein-Barr virus, European Bat Lyssavirus, GB virus C/Hepatitis G virus, Hantaan virus, Hendra virus, Hepatitis A virus, Hepatitis B virus, Hepatitis C virus, Hepatitis delta virus, Human adenovirus, Human astrovirus, Human coronavirus, Human cytomegalovirus, Human enterovirus 68,70, Human herpesvirus 1, Human herpesvirus 2, Human herpesvirus 6, Human herpesvirus 7, Human Immunodeficiency Virus (HIV), Human papillomavirus 1, Human papillomavirus 2, Human papillomavirus 16,18, Human parainfluenza virus, Human parvovirus B19, Human respiratory syncytial virus, Human rhinovirus, Human SARS coronavirus, Human spumarterovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro visu, MERS coronavirus,

Measles virus, Mengo encephalomyocarditis, Merkell cell polyomavirus, Mokola virus, Molluscum contagiosum virus, Monkeypox virus, Mumps virus, Murray valley encephalitis virus, New York virus, Nipah virus, Norwalk virus, O′Nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama, Salivirus A, Sandfly fever sicilian virus, Sapporo virus, SARS coronavirus- 2, Semliki forest virus, Seoul virus, Simian foamy virus, Simian virus 5, Sindbis virus, Southampton virus, St. Louis encephalitis virus, Tick-borne Powassan virus, Torque teno virus, Toscana virus, Uukuniemi virus, Varicella-zoster virus, Variola virus, Venezuelan equine encephalitis virus, WU polyomavirus, Yaba Monkey tumor virus, Yaba-like disease virus, Yellow fever virus, or Zika virus.

48. The method of embodiment 43, wherein the bacterial infection is due to Staphylococcus, Streptococcus, Mycobacterium, Bacillus, Salmonella, Vibrio, Spirochete, Neisseria, Diplococcus, Pseudomonas, Clostridium, Treponaema, Spirillum, Pneumococcus, or combinations thereof.

49. The method of any one of embodiments 42-48, wherein the cytokine release syndrome is due to one or more immune-targeted therapies.

50. The method of embodiment 49, wherein the immune-targeted therapy is an antibody, a protein, a peptide, a cytokine, an immune signaling modulator, an mRNA, an oncolytic virus, or a cell-based therapy.

51. The method of embodiment 50, wherein the antibody is a monoclonal antibody, a polyclonal antibody, a bi-specific antibody, a tri-specific antibody, or a bi-specific T-cell engager (BiTE) antibody.

52. The method of any one of embodiments 50-51, wherein the antibody targets CD2, CD3, CD20, CD27, CD28, CD30, CD4OL, CD137, OX-40, GITR, LIGHT, DR3, SLAM, or ICOS.

53. The method of embodiment 50, wherein the cytokine is IFN-α, IFN-γ, IL-2, IL-10, IL-12, IL-15, IL-15/IL-15Rα, IL-18, IL-21, GM-CSF, or variants thereof.

54. The method of embodiment 50, wherein the immune signaling modulator is IL-1R, IL-2Rα, IL-2Rβ, IL-2Rγ, IL-3Rα, CSF2RB, IL-4R, IL-5Rα, CSF2RB, IL-6Rα, gp130, IL-7Rα, IL-9R, IL-10Rα, IL-10Rβ, IL-12Rβ1, IL-12Rβ2, IL-13Rα1, IL-13Rα2, IL-15Rα, IL-21R, IL23R, IL-27Rα, IL-31Rα, OSMR, CSF-1R, GM-CSF-R, cell-surface IL-15, IL-10Rα, IL-10Rβ, IL-20Rα, IL-20Rβ, IL-22Rα1, IL-22Rα2, IL-22R13, IL-28RA, TLR, JAK, BTK, TYK, SYK, MAPK, PI3K, NFκB, NFAT, STAT, or a kinase.

55. The method of embodiment 50, wherein the cell-based therapy comprises allogenic, autologous, or iPSC-derived T-cells, NK cells, red blood cells, stem cells, antigen presenting cells, macrophages, or dendritic cells.

56. The method of any one embodiments 22-55, wherein administering the negatively charged particles in a subject relieves one or more symptoms of acute inflammation.

57. The method of embodiment 56, wherein the one or more symptoms of acute inflammation are selected from the group consisting respiratory distress, low blood pressure, headache, hypoxemia, respiratory acidosis, hypercapnia, edema, pulmonary edema, alveolar edema, lung damage, liver damage, kidney damage, abnormal liver function, liver dysfunction, increased liver enzymes, multi-organ dysfunction, increased monocytes, increased neutrophils, increased ferritin, decreased lymphocytes, increased neutrophil to lymphocyte ratio (NLR), increased liver enzymes, pancytopenia, coagulopathy, increased levels of d-dimer, decreased PaO2/FiO2, decreased SpO2/FiO2, or increased levels of pro-inflammatory molecules.

58. The method of embodiment 57, wherein the pro-inflammatory molecules are selected from the group consisting of IL-1β, IL-2, IL-6, IL-7, IL-8, IL-10, IL-33, TNF-α, IFN-γ, IP-10, MIP-1β, MCP-1, GM-CSF, c-reactive protein (CRP), and sST.

59. The method of embodiment 57, wherein the abnormal liver function is determined by assessing levels of aspartate aminotransferase (AST), alanine aminotransferase (ALT), alkaline phosphatase (ALP), albumin and total protein, bilirubin, gamma-glutamyltransferase (GGT), or lactate dehydrogenase (LD).

60. The method of any one of embodiments 22-59, wherein administering negatively charged particles in a subject suffering from acute inflammation reduces the risk of mortality.

61. The method of any one of embodiments 22-60, wherein the administering of the negatively charged particles in a subject suffering from acute inflammation improves recovery.

62. The method of any one of embodiments 22-61, wherein administering negatively charged particles normalizes pro-inflammatory cytokines and chemokines, c-reactive protein, d-dimer, liver enzymes, ferritin, monocytes, neutrophils, macrophages, lymphocytes, aspartate aminotransferase (AST), alanine aminotransferase (ALT), alkaline phosphatase (ALP), albumin and total protein, bilirubin, gamma-glutamyltransferase (GGT), and lactate dehydrogenase (LD) levels in blood.

63. The method of embodiments 22-62, wherein administering negatively charged particles normalizes lung function.

64. The method of embodiment 63, wherein lung function is assessed using PaO2/FiO2, SpO2/FiO2, CT, X-ray, bronchoscopy, PET, and MRI.

65. The method of embodiments 63 or 64, wherein administering negatively charged particles reduces the use of supplemental oxygen therapy, high-flow oxygen, mechanical ventilation, CPAP, organ support, pressors, RRT, ECMO, and steroids.

66. The method of any one of embodiments 22-65, wherein administering negatively charged particles reduces hospitalization days.

67. A method of treating acute inflammation in hospitalized adults with systemic inflammation, sepsis, or pneumonia associated with respiratory viral infections in a subject in need thereof, comprising administering negatively charged particles that are free from attached or encapsulated drug, wherein the negatively charged particles comprise one or more biodegradable, pharmaceutically acceptable polymers; and particles are administered at a dose of between 1 mg/kg to 10 mg/kg.

68. The method of embodiment 67, wherein respiratory viral infection is associated with influenza or SARS-CoV-2.

69. The method of any one of embodiments 67-68, wherein the negatively charged particles are administered at a dose level of between 1 mg/kg to 6 mg/kg.

70. The method of any one of embodiments 67-69, wherein the negatively charged particles are administered at a dose level of 5 mg/kg.

71. The method of any one of embodiments 67-70, wherein the negatively charged particles are administered at a dose level of between 50 mg to 400 mg.

72. The method of any one of embodiments 67-71, wherein the negatively charged particles comprise one or more biodegradable polymers.

73. The method of any one of embodiments 67-72, wherein the negatively charged particles comprise Poly (lactic-co-glycolic acid) (PLGA), poly (lactic acid) (PLA), poly (glycolic acid) (PGA), or polystyrene.

74. The method of any one of embodiments 67-73, wherein the negatively charged particles comprise PLGA.

75. The method of any one of embodiments 67-74, wherein the negatively charged particles have a zeta potential is between about −30 mV and −80 mV.

76. The method of any one of embodiments 67-75, wherein the negatively charged particles have an average diameter between 0.3 μm and 3 μm, or between 0.3 μm and

77. The method of any one of embodiments 67-76, wherein administering the negatively charged particles in a subject in need thereof leads to a reduction from baseline in the levels of serum c-reactive protein.

78. The method of embodiment 77, wherein the levels of serum c-reactive protein are reduced by 2, 5, 10, 15, 20, 25, 30, 35, 40, 45, 50, 55, 60, 65, 70, 75, 80, 85, 90, 95, or 100-fold compared to baseline.

79. The method of any one of embodiments 67-78, wherein administering the negatively charged particles in a subject in need thereof leads to a reduction from baseline in the levels of serum ferritin.

80. The method of embodiment 79, wherein the levels of serum ferritin are reduced by 2, 5, 10, 15, 20, 25, 30, 35, 40, 45, 50, 55, 60, 65, 70, 75, 80, 85, 90, 95, or 100-fold compared to baseline.

81. The method of any one of embodiments 67-80, wherein administering the negatively charged particles in a subject in need thereof leads to a reduction from baseline in the levels of serum d-dimer.

82. The method of embodiment 81, wherein the levels of serum d-dimer protein are reduced by 2, 5, 10, 15, 20, 25, 30, 35, 40, 45, 50, 55, 60, 65, 70, 75, 80, 85, 90, 95, or 100-fold compared to baseline.

83. The method of any one of embodiments 67-82, wherein administering the negatively charged particles in a subject in need thereof leads to a reduction from baseline in the number of neutrophils in blood.

84. The method of embodiment 83, wherein the number of neutrophils in blood are reduced by 2, 5, 10, 15, 20, 25, 30, 35, 40, 45, 50, 55, 60, 65, 70, 75, 80, 85, 90, 95, or 100-fold compared to baseline.

85. The method of any one of embodiments 67-84, wherein administering the negatively charged particles in a subject in need thereof leads to an increase from baseline in the number of lymphocytes in blood.

86. The method of embodiment 85, wherein the number of lymphocytes in blood are increased by 2, 5, 10, 15, 20, 25, 30, 35, 40, 45, 50, 55, 60, 65, 70, 75, 80, 85, 90, 95, or 100-fold compared to baseline.

87. The method of any one of embodiments 67-86, wherein administering the negatively charged particles in a subject in need thereof leads to a decrease from baseline in the neutrophil to lymphocyte ratio (NLR).

88. The method of embodiment 87, wherein administering the negatively charged particles in a subject in need thereof leads to an NLR of <4.

89. The method of any one of embodiments 67-88, wherein administering the negatively charged particles in a subject in need thereof leads to an increase from baseline in an SpO2/FiO2 ratio.

90. The method of embodiment 89, wherein administering the negatively charged particles in a subject in need thereof leads to a SpO2/FiO2 ratio of >300 mmHg.

91. The method of any one of embodiments 67-90, wherein the administering of the negatively charged particles in a subject in need thereof leads to a reduction in the number of hospitalization days.

92. The method of any one of embodiments 67-91, wherein the administering of the negatively charged particles in a subject in need thereof leads to an increase in the number of ventilator-free days.

93. The method of any one of embodiments 67-92, wherein the administering of the negatively charged particles in a subject in need thereof leads to a reduced risk of mortality.

94. The method of any one of embodiments 67-93, wherein administering the negatively charged particles in a subject in need thereof leads to improved lung function compared to baseline.

95. A method of treating cytokine storm syndrome (CSS) in a subject, comprising administering to the subject surface-functionalized particles having a negative zeta potential, wherein the surface-functionalized particles are free from another therapeutic agent.

96. The method of embodiment 95, wherein the subject suffers from one or more conditions selected from: a viral infection, a bacterial infection, sepsis, cytokine release syndrome (CRS), severe inflammatory response syndrome (SIRS), hypercytokinemia, macrophage activation syndrome (MAS), reactive hemophagocytic syndrome, secondary hemophagocytic lymphohistiocytosis (sHLH), or a traumatic injury.

97. The method of any one of embodiments 95-96, wherein administering the surface-functionalized particles in the subject reduces one or more symptoms of CSS.

98. The method of embodiment 97, wherein the symptom is selected from one or more of: multi-organ dysfunction, brain damage, lung damage, liver damage, kidney damage, heart damage, edema, cerebral edema, pulmonary edema, alveolar edema, respiratory distress, hypoxemia, respiratory acidosis, hypertriglyceridemia, leukopenia, cytopenia, or elevated levels of inflammatory markers.

99. The method of embodiment 98, wherein the inflammatory marker is IL-1β, IL-2, IL-6, IL-8, TNF-α, IFN-γ, MCP-1, c-reactive protein, or ferritin.

100. The method of embodiment 96, wherein the viral infection is due to one or more of: a DNA virus, an RNA virus, or a retrovirus.

101. The method of embodiment 100, wherein the DNA virus is a single-stranded DNA (ssDNA) virus, or a double-stranded (dsDNA) virus, and the RNA virus is a double-stranded RNA virus, a single-stranded RNA (ssRNA) (+) virus, ssRNA (−) virus, or a circular ssRNA virus.

102. The method of any one of embodiments 100-101, wherein the virus is a respiratory virus.

103. The method of any one of embodiments 100-102, wherein the virus is selected from: Adeno-associated virus, Aichi virus, Australian Bat Lyssavirus, BK polyomavirus, Banna virus, Barmah forest virus, Bunyavirus snowshoe hare, Cercopithecine herpes virus, Chandipura virus, Chikungunya virus, Cosavirus A, Cowpox virus, Coxsackievirus, Crimean-Congo hemorrhagic fever virus, Coronavirus, Dengue virus, Dhori virus, Dugbe virus, Duvenhage virus, Eastern equine encephalitis virus, Echovirus, Encephalomyocarditis virus, Epstein-Barr virus, European Bat Lyssavirus, GB virus C/Hepatitis G virus, Hantaan virus, Hendra virus, Hepatitis A virus, Hepatitis B virus, Hepatitis C virus, Hepatitis delta virus, Human adenovirus, Human astrovirus, Human coronavirus, Human cytomegalovirus, Human enterovirus 68,70, Human herpesvirus 1, Human herpesvirus 2, Human herpesvirus 6, Human herpesvirus 7, Human Immunodeficiency Virus (HIV), Human papillomavirus 1, Human papillomavirus 2, Human papillomavirus 16,18, Human parainfluenza virus, Human parvovirus B19, Human respiratory syncytial virus, Human rhinovirus, Human SARS coronavirus, Human spumarterovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro visu, MERS coronavirus, Measles virus, Mengo encephalomyocarditis, Merkell cell polyomavirus, Mokola virus, Molluscum contagiosum virus, Monkeypox virus, Mumps virus, Murray valley encephalitis virus, New York virus, Nipah virus, Norwalk virus, O'Nyong-nyong virus, Orf virus, Oropouche virus, Pichinde virus, Poliovirus, Punta toro phlebovirus, Puumala virus, Rabies virus, Rift valley fever virus, Rotavirus A, Ross river virus, Rotavirus A, Rotavirus B, Rotavirus C, Rubella virus, Sagiyama, Salivirus A, Sandfly fever sicilian virus, Sapporo virus, SARS coronavirus-2, Semliki forest virus, Seoul virus, Simian foamy virus, Simian virus 5, Sindbis virus, Southampton virus, St. Louis encephalitis virus, Tick-borne Powassan virus, Torque teno virus, Toscana virus, Uukuniemi virus, Varicella-zoster virus, Variola virus, Venezuelan equine encephalitis virus, WU polyomavirus, Yaba Monkey tumor virus, Yaba-like disease virus, Yellow fever virus, or Zika virus.

104. The method of any one of embodiments 96-103, wherein the bacterial infection is due to Staphylococcus, Streptococcus, Mycobacterium, Bacillus, Salmonella, Vibrio, Spirochete, Neisseria, Diplococcus, Pseudomonas, Clostridium, Treponaema, Spirillum, or combinations thereof.

105. The method of any one of embodiments 96-104, wherein the CRS is due to one or more immune-targeted therapies.

106. The method of embodiment 105, wherein the immune-targeted therapy is an antibody, a protein therapeutic, a peptide, a cytokine, an immune signaling modulator, an mRNA, an oncolytic virus, or a cell-based therapy.

107. The method of embodiment 106, where the antibody is a monoclonal antibody, a polyclonal antibody, a bi-specific antibody, a tri-specific antibody, or a bi-specific T-cell engager (BiTE) antibody.

108. The method of any one of embodiments 106-107, wherein the antibody targets one or more of: CD2, CD3, CD20, CD27, CD28, CD30, CD4OL, CD137, OX-40, GITR, LIGHT, DR3, SLAM, or ICOS.

109. The method of any one of embodiments 106-108, wherein the cytokine is selected from IFN-α, IFN-γ, IL-2, IL-10, IL-12, IL-15, IL-15/IL-15Rα, IL-18, IL-21, GM-CSF, or variants thereof.

110. The method of any one of embodiments 106-109, wherein the immune signaling modulator targets one or more of: IL-1R, IL-2Rα, IL-2Rβ, IL-2Rγ, IL-3Rα, CSF2RB, IL-4R, IL-5Rα, CSF2RB, IL-6Rα, gp130, IL-7Rα, IL-9R, IL-10Rα, IL-10Rβ, IL-12R131, IL-12Rβ2, IL-13Rα1, IL-13Rα2, IL-15Rα, IL-21R, IL23R, IL-27Rα, IL-31Rα, OSMR, CSF-1R, GM-CSF-R, cell-surface IL-15, IL-10Rα, IL-10Rβ, IL-20Rα, IL-20Rβ, IL-22Rα1, IL-22Rα2, IL-22Rβ, IL-28RA, TLR, JAK, BTK, TYK, SYK, MAPK, PI3K, NFκB, NFAT, STAT, or a kinase.

111. The method of any one of embodiments 106-110, wherein the cell-based therapy comprises allogenic, autologous, or iPSC-derived cells.

112. The method of any one of embodiments 106-111, wherein the cell-based therapy comprises one or more of: T-cells, NK-cells, red blood cells, stem cells, antigen-presenting cells, macrophages, or dendritic cells.

113. The method of any one of embodiments 95-112, wherein the surface-functionalized particle is surface functionalized by an addition of one or more carboxyl groups to the surface of the particle.

114. The method of any one of embodiments 95-113, wherein the surface-functionalized particle has a zeta potential between about −100 mV and about −1 mV.

115. The method of embodiment 114, wherein the surface-functionalized particle has a zeta potential between about −80 mV and about −30 mV.

116. The method of any one of embodiments 95-115, wherein the average diameter of the surface-functionalized particle is between about 0.1 μm to about 10 μm.

117. The method of embodiment 116, wherein the average diameter of the surface-functionalized particle is between about 400 nm to about 800 nm.

118. The method of any one of embodiments 95-117, wherein the particle comprises one or more of: polyglycolic acid (PGA), polylactic acid (PLA), polystyrene, poly(lactic-co-glycolic acid) (PLGA), chitosan, polysaccharide, a lipid, diamond, iron, zinc, cadmium, gold, or silver.

119. The method of embodiment 118, wherein the surface-functionalized particles are poly(lactic-co-glycolic acid) (PLGA) particles.

120. The method of embodiment 119, wherein the PLGA particles comprises a ratio of poly lactic acid:poly glycolic acid ranging from about 90:10 to about 10:90, from about 50:50 to about 90:10, about 50:50 to about 80:20; from about 90:10 to about 50:50, or from about 80:20 to about 50:50.

121. The method of embodiment 120, wherein the particle comprises 50:50 poly lactic acid:poly glycolic acid.

122. The method of any of embodiments 95-121, wherein the surface-functionalized particles are administered intravenously.

### REFERENCES

1 Thompson B T, Chambers R C, Liu K D. Acute Respiratory Distress Syndrome. N Engl J Med. 2017;377(6):562-572.
2. Han S, Mallampalli R K. The acute respiratory distress syndrome: from mechanism to translation. J Immunol. 2015;194(3):855-860.
3. Matthay M A, Zemans R L, Zimmerman G A, et al. Acute respiratory distress syndrome. Nature Reviews Disease Primers. 2019;5(1):18.
4. Fajgenbaum D C, June C H. Cytokine Storm. New England Journal of Medicine. 2020;383(23):2255-2273.
5. Behrens E M, Koretzky G A. Review: Cytokine Storm Syndrome: Looking Toward the Precision Medicine Era. Arthritis Rheumatol. 2017;69(6):1135-1143.
6. Schulert G S, Grom A A. Macrophage activation syndrome and cytokine-directed therapies. Best Pract Res Clin Rheumatol. 2014;28(2):277-292.
7 Tisoncik J R, Korth M J, Simmons C P, Farrar J, Martin T R, Katze M G. Into the eye of the cytokine storm. Microbiol Mol Biol Rev. 2012;76(1):16-32.
8. Norelli M, Camisa B, Barbiera G, et al. Monocyte-derived IL-1 and IL-6 are differentially required for cytokine-release syndrome and neurotoxicity due to CAR T cells. Nat Med. 2018;24(6):739-748.
9. Sánchez A, Mejia S P, Orozco J. Recent Advances in Polymeric Nanoparticle-Encapsulated Drugs against Intracellular Infections. Molecules. 2020;25(16).
10. Zielińska A, Carreiró F, Oliveira A M, et al. Polymeric Nanoparticles: Production, Characterization, Toxicology and Ecotoxicology. Molecules. 2020;25(16).
11. A minimal common outcome measure set for COVID-19 clinical research. Lancet Infect Dis. 2020;20(8):e192-e197.
12. Fong T G, Fearing M A, Jones RN, et al. Telephone interview for cognitive status: Creating a crosswalk with the Mini-Mental State Examination. Alzheimers Dement. 2009;5(6):492-497.
13. Force* TADT. Acute Respiratory Distress Syndrome: The Berlin Definition. JAMA. 2012;307(23):2526-2533.
14. The 2019 revision of the International Standards for Neurological Classification of Spinal Cord Injury (ISNCSCI)-What's new? Spinal Cord. 2019;57(10):815-817.
15. Fardet L, Galicier L, Lambotte 0, et al. Development and validation of the HScore, a score for the diagnosis of reactive hemophagocytic syndrome. Arthritis Rheumatol. 2014;66(9):2613-2620.
16. Villar J, Kacmarek R M. The APPS: an outcome score for the acute respiratory distress syndrome. J Thorac Dis. 2016;8(10):E1343-e1347.
17. Thomas KS, Dosa D, Wysocki A, Mor V. The Minimum Data Set 3.0 Cognitive Function Scale. Med Care. 2017;55(9):e68-e72.

What is claimed is:

1. A method of treating acute respiratory distress syndrome (ARDS) or cytokine storm syndrome (CSS) in a subject comprising administering to the subject a therapeutically effective amount of negatively charged particles, wherein the negatively charged particles are free from other therapeutically active agents.

2. The method of claim 1, wherein the ARDS or the CSS are the result of direct lung-injury or indirect lung-injury.

3. The method of claim 1, wherein the ARDS or the CSS are the result of pneumonia, lung inflammation, bacterial infection, viral infection, fungal infection, opportunistic infection, sepsis, aspiration of gastric contents, traumatic injury, burn injury, pancreatitis, pulmonary contusion, hemorrhagic shock, near drowning, blood transfusion, inhalation injury, cytokine release syndrome (CRS), severe inflammatory response syndrome (SIRS), hypercytokinemia, macrophage activation syndrome (MAS), reactive hemophagocytic syndrome, secondary hemophagocytic lymphohistiocytosis (sHLH), a traumatic injury, or combinations thereof.

4. The method of claim 3, wherein the ARDS or the CSS are the result of the viral infection, and wherein the viral infection is due to a DNA virus, an RNA virus, or a retrovirus.

5. The method of claim 4, wherein the DNA virus is a single-stranded DNA (ssDNA) virus, or a double-stranded (dsDNA) virus, and wherein the RNA virus is a double-stranded RNA virus, a single-stranded RNA (ssRNA) (+) virus, ssRNA (−) virus, or a circular ssRNA virus.

6. The method of claim 4 or 5, wherein the DNA virus, the RNA virus, or the retrovirus is a respiratory virus.

7. The method of any one of claims 4-6, wherein the DNA virus, the RNA virus, or the retrovirus is selected from the group consisting of: Adeno- associated virus, Aichi virus, Australian Bat Lyssavirus, BK polyomavirus, Banna virus, Barmah forest virus, Bunyavirus snowshoe hare, Cercopithecine herpes virus, Chandipura virus, Chikungunya virus, Cosavirus A, Cowpox virus, Coxsackievirus, Crimean-Congo hemorrhagic fever virus, Coronavirus, Dengue virus, Dhori virus, Dugbe virus, Duvenhage virus, Eastern equine encephalitis virus, Echovirus, Encephalomyocarditis virus, Epstein- Barr virus, European Bat Lyssavirus, GB virus C/Hepatitis G virus, Hantaan virus, Hendra virus, Hepatitis A virus, Hepatitis B virus, Hepatitis C virus, Hepatitis delta virus, Human adenovirus, Human astrovirus, Human coronavirus, Human cytomegalovirus, Human enterovirus 68,70, Human herpesvirus 1, Human herpesvirus 2, Human herpesvirus 6, Human herpesvirus 7, Human Immunodeficiency Virus (HIV), Human papillomavirus 1, Human papillomavirus 2, Human papillomavirus 16,18, Human parainfluenza virus, Human parvovirus B19, Human respiratory syncytial virus, Human rhinovirus, Human SARS coronavirus, Human spumarterovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunj in virus, Lagos bat virus, Lake Victoria Marburgvirus, Langat virus, Lassa virus, Lordsdale virus, Louping ill virus, Lymphocytic choriomeningitis virus, Machupo virus, Mayaro visu, MERS coronavirus, Measles virus, Mengo encephalomyocarditis, Merkell cell polyomavirus, Mokola virus, Molluscum contagiosum virus, Monkeypox virus, Mumps virus, Murray valley encephalitis virus, New York virus, Nipah virus, Norwalk virus, O'Nong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama, Salivirus A, Sandfly fever sicilian virus, Sapporo virus, SARS coronavirus-2, Semliki forest virus, Seoul virus, Simian foamy virus, Simian virus 5, Sindbis virus, Southampton virus, St. Louis encephalitis virus, Tick- borne Powassan virus, Torque teno virus, Toscana virus, Uukuniemi virus, Varicella-zoster virus, Variola virus, Venezuelan equine encephalitis virus, WU polyomavirus, Yaba Monkey tumor virus, Yaba-like disease virus, Yellow fever virus, and Zika virus.

8. The method of claim 3, wherein the ARDS or the CSS are the result of the bacterial infection, and wherein the bacterial infection is due to Staphylococcus, Streptococcus, Mycobacterium, Bacillus, Salmonella, Vibrio, Spirochete, Neisseria, Diplococcus, Pseudomonas, Clostridium, Treponaema, Spirillum, or combinations thereof.

9. The method of claim 1, wherein the ARDS or the CSS are due to one or more immune-targeted therapies.

10. The method of claim 9, wherein the one or more immune-targeted therapies is an antibody, a protein therapeutic, a peptide, a cytokine, an immune signaling modulator, an mRNA, an oncolytic virus, or a cell-based therapy.

11. The method of claim 10, wherein the one or more immune-targeted therapies comprise the antibody, and wherein the antibody is a monoclonal antibody, a polyclonal antibody, a bi-specific antibody, a tri-specific antibody, or a bi-specific T-cell engager (BiTE) antibody.

12. The method of claim 11, wherein the antibody targets CD2, CD3, CD20, CD27, CD28, CD30, CD4OL, CD137, OX-40, GITR, LIGHT, DR3, SLAM, ICOS, LILRB2, LILRB3, LILRB4, PD-1, PD-L1, CTLA-4, IL-12, or IL-15,RTK, EGFR, VEGF, VEGFR, PDGF, PDGFR, HER2/Neu, ER, PR, TGF-β1, TGF-β2, TGF-β3, SIRP-α, PD-1, PD-L1, CTLA-4, CD3, CD25, CD19, CD20, CD39, CD47, CD73, FAP, IL-1β, IL-2R, IL-12, IL-15, IL-15R, IL-23, IL-33, IL-2R, IL-4RαT-cells, B-cells, NK cells, macrophages, monocytes, or neutrophils.

13. The method of claim 10, wherein the one or more immune-targeted therapies comprise the cytokine, and wherein the cytokine is selected from IFN-α, IFN-γ, IL-2, IL-10, IL-12, IL-15, IL-15/IL-15Rα, IL-18, IL-21, GM-CSF, or variants thereof.

14. The method of claim 10, wherein the one or more immune-targeted therapies comprise the immune signaling modulator, and wherein the immune signaling modulator targets one or more of: IL- 1R, IL-2Rα, IL-2Rα, IL-2Rγ, IL-3Rα, CSF2RB, IL-4R, IL-5Rα, CSF2RB, IL-6Rα, gp130, IL-7Rα, IL-9R, IL-10Rα, IL-10Rα, IL-12Rα1, IL-12Rα2, IL-13Rα1, IL-13Rα2, IL-15Rα, IL- 21R, IL23R, IL-27Rα, IL-31Rα, OSMR, CSF-1R, GM-CSF-R, cell-surface IL-15, IL-10Rα, IL-10Rα, IL-20Rα, IL-20Rα, IL-22Rα1, IL-22Rα2, IL-22Rα, IL-28RA, TLR, JAK, BTK, TYK, SYK, MAPK, PI3K, NFKB, NFAT, STAT, or a kinase.

15. The method of claim 10, wherein the one or more immune-targeted therapies comprise the cell-based therapy, and wherein the cell-based therapy comprises allogenic, autologous, or iPSC-derived cells.

16. The method of claim 10, wherein the one or more immune-targeted therapies comprise the cell-based therapy, and wherein the cell-based therapy comprises one or more of: T- cells, NK-cells, red blood cells, stem cells, antigen-presenting cells, macrophages, or dendritic cells.

17. The method of any one of the preceding claims, wherein the negatively charged particles comprise one or more biodegradable polymers.

18. The method of any one of claims 1-17, wherein the negatively charged particles comprise one or more of: polyglycolic acid (PGA), polylactic acid (PLA), polystyrene, poly (lactic-co-glycolic acid) (PLGA), chitosan, polysaccharide, a lipid, diamond, iron, zinc, cadmium, gold, or silver.

19. The method of claim 18, wherein the negatively charged particles comprise the PLGA.

20. The method of claim 18, wherein the negatively charged particles that comprise PLGA comprise a ratio of poly lactic acid:poly glycolic acid ranging from about 90:10 to about 10:90, from about 50:50 to about 90:10, about 50:50 to about 80:20; from about 90:10 to about 50:50, or from about 80:20 to about 50:50.

21. The method of claim 20, wherein the negatively charged particles that comprise the PLGA comprise the ratio of poly lactic acid: poly glycolic acid ranging from about 50:50.

22. The method of any one of claims 1-21, wherein the negatively charged particles further comprise carboxyl groups on a surface.

23. The method of any one of claims 1-22, wherein the negative zeta potential ranges from about −100 mV and about −1 mV.

24. The method of claim 23, wherein the negative zeta potential ranges from about −80 mV and about −30 mV.

25. The method of any one of claims 1-24, wherein the negatively charged particles have a mean diameter ranging from about 0.1 μm to about 10 μm.

26. The method of claim 25, wherein the mean diameter of the negatively charged particle ranges from about 300 nm to about 800 nm.

27. The method of any one of claims 1-26, wherein the administering of the negatively charged particles to the subject improves one or more symptoms associated with the ARDS or CSS.

28. The method of claim 27, wherein the one or more symptoms associated with the ARDS or the CSS are selected from the group consisting of lung inflammation, atelectasis, distressed breathing, fatigue, low blood pressure, fever, headache, hypoxemia, respiratory acidosis, hypercapnia, edema, pulmonary edema, alveolar edema, multi-organ dysfunction, brain damage, lung damage, liver damage, kidney damage, heart damage, edema, cerebral edema, pulmonary edema, alveolar edema, respiratory distress, hypoxemia, respiratory acidosis, hypertriglyceridemia, leukopenia, cytopenia, and elevated levels of inflammatory markers.

29. The method of claim 28, wherein the inflammatory markers consist of IL-1β, IL-2, IL-6, IL-7, IL-8, IL-10, TNF-α, IFN-γ, IP-10, MIP-1β, MCP-1, GM-CSF, c-reactive protein, d-dimer, ferritin, neutrophil extracellular traps (NETs), and combinations thereof.

30. The method of any one of claims 1-29, wherein the administering of the negatively charged particles reduces accumulation of inflammatory mediators in circulation or at sites of inflammation.

31. The method of any of the preceding claims, wherein the negatively charged particles are administered intravenously.

32. A method of treating acute inflammation in hospitalized adults with systemic inflammation, sepsis, or pneumonia associated with respiratory viral infections in a subject in need thereof, comprising administering negatively charged particles that are free from attached or encapsulated drug, wherein the negatively charged particles comprise one or more biodegradable, pharmaceutically acceptable polymers; and particles are administered at a dose of between 1 mg/kg to 10 mg/kg.

33. The method of claim 32, wherein respiratory viral infection is associated with influenza and SARS-CoV-2.

34. The method of any one of claims 32-33, wherein the negatively charged particles are administered at a dose level of between 1 mg/kg to 6 mg/kg.

35. The method of any one of claims 32-34, wherein the negatively charged particles are administered at a dose level of 5 mg/kg.

36. The method of any one of claims 32-35, wherein the negatively charged particles are administered at a dose level of between 50 mg to 400 mg.

37. The method of any one of claims 32-36, wherein the negatively charged particles comprise one or more biodegradable polymers.

38. The method of any one of claims 32-37, wherein the negatively charged particles comprise Poly (lactic-co-glycolic acid) (PLGA), poly (lactic acid) (PLA), poly (glycolic acid) (PGA), or polystyrene.

39. The method of any one of claims 32-38, wherein the negatively charged particles comprise PLGA.

40. The method of any one of claims 32-39, wherein the negatively charged particles have a zeta potential is between about −30 mV and -80 mV.

41. The method of any one of claims 32-40, wherein the negatively charged particles have an average diameter between 0.3 μm and 3 μm, or between 0.3 μm and 1 μm.

42. The method of any one of claims 32-41, wherein the administering of the negatively charged particles to the subject in need thereof leads to a reduction from baseline in the levels of serum c-reactive protein.

43. The method of claim 42, wherein the levels of serum c-reactive protein are reduced by 2, 5, 10, 15, 20, 25, 30, 35, 40, 45, 50, 55, 60, 65, 70, 75, 80, 85, 90, 95, or 100-fold compared to baseline.

44. The method of any one of claims 32-43, wherein the administering of the negatively charged particles to the subject in need thereof leads to a reduction from baseline in the levels of serum ferritin.

45. The method of claim 44, wherein the levels of the serum ferritin are reduced by 2, 5, 10, 15, 20, 25, 30, 35, 40, 45, 50, 55, 60, 65, 70, 75, 80, 85, 90, 95, or 100-fold compared to baseline.

46. The method of any one of claims 32-45, wherein the administering of the negatively charged particles to the subject in need thereof leads to a reduction from baseline in the levels of serum d-dimer.

47. The method of claim 46, wherein the levels of the serum d-dimer protein are reduced by 2, 5, 10, 15, 20, 25, 30, 35, 40, 45, 50, 55, 60, 65, 70, 75, 80, 85, 90, 95, or 100-fold compared to baseline.

48. The method of any one of claims 32-47, wherein the administering of the negatively charged particles to the subject in need thereof leads to a reduction from baseline in the number of neutrophils in blood.

49. The method of claim 48, wherein the number of neutrophils in the blood are reduced by 2, 5, 10, 15, 20, 25, 30, 35, 40, 45, 50, 55, 60, 65, 70, 75, 80, 85, 90, 95, or 100-fold compared to baseline.

50. The method of any one of claims 32-49, wherein the administering of the negatively charged particles to the subject in need thereof leads to an increase from baseline in the number of lymphocytes in blood.

51. The method of claim 50, wherein the number of lymphocytes in the blood are increased by 2, 5, 10, 15, 20, 25, 30, 35, 40, 45, 50, 55, 60, 65, 70, 75, 80, 85, 90, 95, or 100-fold compared to baseline.

52. The method of any one of claims 32-51, wherein the administering of the negatively charged particles to the subject in need thereof leads to a decrease from baseline in the neutrophil to lymphocyte ratio (NLR).

53. The method of claim 52, wherein the administering of the negatively charged particles to the subject in need thereof leads to an NLR of ≤4.

54. The method of any one of claims 32-53, wherein the administering of the negatively charged particles to the subject in need thereof leads to an increase from baseline in a SpO₂/FiO₂ ratio.

55. The method of claim 54, wherein the SpO₂/FiO₂ ratio if ≥300 mmHg.

56. The method of any one of claims 32-55, wherein the administering of the negatively charged particles to the subject in need thereof leads to a reduction in the number of hospitalization days.

57. The method of any one of claims 32-56, wherein the administering of the negatively charged particles to the subject in need thereof leads to an increase in the number of ventilator-free days.

58. The method of any one of claims 32-57, wherein the administering of the negatively charged particles to the subject in need thereof leads to a reduced risk of mortality.

59. The method of any one of claims 32-58, wherein the administering of the negatively charged particles to the subject in need thereof leads to improved lung function compared to baseline.
